(19) United States
(12) Patent Application Publication
(10) Pub. No.: US 2004/0126401 A1
(43) Pub. Date: Jul. 1, 2004

(54) Cosmetic composition comprising a mixture of polymers

(76) Inventor: Nathalie Collin, Sceaux (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/466,166

(22) PCT Filed: Jan. 14, 2002
(86) PCT No.: PCT/FR02/00129

(30) Foreign Application Priority Data
Jan. 15, 2001 (FR) 01/00479

(51) Int. Cl. A61K 7/06; A61K 7/11

(52) U.S. Cl. 424/401; 424/70.17

(57) ABSTRACT

The invention relates to a composition comprising, in a physiologically acceptable medium containing a fatty phase:

(i) a first polymer with a weight-average molecular mass of less than 100 000, comprising a) a polymer skeleton with hydrocarbon-based repeating units containing at least one hetero atom, and optionally b) optionally functionalized pendent and/or terminal fatty chains containing from 6 to 120 carbon atoms, which are linked to these hydrocarbon-based units,

(ii) an anionic film-forming polymer,

(iii) a cationic film-forming polymer.

The composition produces a fast makeup result on keratin materials.

Application to making up and caring for keratin materials, especially as a mascara.

The present invention relates to a makeup composition comprising a mixture of particular polymers to obtain a rapid makeup result on keratin materials. The use according to the invention is more particularly intended for keratin fibres, especially substantially longilinear human keratin fibres such as the eyelashes, the eyebrows and the hair, or alternatively false eyelashes or wigs. More especially, the composition is a mascara. The term “mascara” means a makeup composition for the eyelashes, a makeup base, a product to be applied over a makeup, also known as a topcoat, or a cosmetic treatment product for the eyelashes.

Compositions for coating the eyelashes, known as mascara, generally comprise, in a known manner, at least one wax and at least one film-forming polymer to deposit a makeup film on the eyelashes and coat them, for example as described in documents WO-A-91/12793 and WO-A-95/15741. Users expect these products to have good cosmetic properties, such as adhesion to the eyelashes, lengthening or curling of the eyelashes, or alternatively good staying power of the mascara over time, in particular good resistance to rubbing, for example with the fingers or fabrics (handkerchiefs or towels). A mascara containing waxes and a combination of anionic polymer and of cationic polymer is especially known from document FR-A-2 528 699.

However, with these compositions, the makeup properties, for instance the coating, lengthening or curling of the eyelashes, are obtained when a large amount of product is applied to the eyelashes using an applicator, such as a mascara brush. The user must thus apply the brush impregnated with product onto the eyelashes several times, which obliges the user to devote a certain amount of time to applying the makeup and to obtaining the desired makeup results. However, users who are in a hurry may perceive this time as being far too long. There is thus a need to provide mascaras that can quickly and easily produce the expected makeup result.

The aim of the present invention is to provide a composition for making up keratin materials, especially keratin fibres such as the eyelashes, which applies easily to the keratin materials and quickly produces a makeup result that has good cosmetic properties.

The inventors have found, surprisingly, that the use of a nonionic polymer containing a particular hetero atom in a cosmetic composition containing an anionic polymer and a cationic polymer can improve the adhesion properties of the composition to keratin materials, especially to keratin fibres such as the eyelashes. The composition applies easily to the keratin materials and can be quickly deposited in an amount that is sufficient to produce a makeup result having the expected cosmetic properties. In particular, a thick deposit of the makeup is quickly obtained on the keratin materials, which avoids the users having to spend too long applying the composition to the keratin materials. The composition thus allows a fast makeup (or “express” makeup) of keratin materials.

Thus, for a mascara, a makeup that quickly thickens the keratin fibres, especially the eyelashes, is obtained; instantaneous loading of the eyelashes is thus observed.

More specifically, one subject of the invention is a composition, especially a makeup or care composition for keratin materials, comprising, in a physiologically acceptable medium containing a fatty phase:

(i) a first polymer with a weight-average molecular mass of less than 100 000, comprising a) a polymer skeleton with hydrocarbon-based repeating units containing at least one hetero atom, and optionally b) optionally functionalized pendent and/or terminal fatty chains containing from 6 to 120 carbon atoms, which are linked to these hydrocarbon-based units,

(ii) an anionic film-forming polymer,

(iii) a cationic film-forming polymer.

A subject of the invention is also a non-therapeutic cosmetic makeup or care process for keratin materials, especially keratin fibres such as the eyelashes, comprising the application to the said keratin materials of a composition as defined above.

A subject of the invention is also the use of

(i) a first polymer with a weight-average molecular mass of less than 100 000, comprising a) a polymer skeleton with hydrocarbon-based repeating units containing at least one hetero atom, and optionally b) optionally functionalized pendent and/or terminal fatty chains containing from 6 to 120 carbon atoms, which are linked to these hydrocarbon-based units,

(ii) an anionic film-forming polymer,

(iii) a cationic film-forming polymer, in a makeup composition for keratin materials, comprising a physiologically acceptable medium containing a fatty phase, to obtain a fast makeup result on the keratin materials.

A subject of the invention is also a cosmetic process for quickly making up keratin materials, which consists in introducing into a cosmetic makeup composition comprising a fatty phase:

(i) a first polymer with a weight-average molecular mass of less than 100 000, comprising a) a polymer skeleton with hydrocarbon-based repeating units containing at least one hetero atom, and optionally b) optionally functionalized pendent and/or terminal fatty chains containing from 6 to 120 carbon atoms, which are linked to these hydrocarbon-based units,

(ii) an anionic film-forming polymer,

(iii) a cationic film-forming polymer.

A subject of the invention is also a cosmetic process for increasing the adhesion and/or the fast loading of a cosmetic makeup composition, which consists in introducing into the said composition containing a fatty phase:

(i) a first polymer with a weight-average molecular mass of less than 100 000, comprising a) a polymer skeleton with hydrocarbon-based repeating units containing at least one hetero atom, and optionally b) optionally functionalized pendent and/or terminal fatty chains containing from 6 to 120 carbon atoms, which are linked to these hydrocarbon-based units,

(ii) an anionic film-forming polymer,

(iii) a cationic film-forming polymer.

The expression “physiologically acceptable medium” means a non-toxic medium that may be applied to the skin, the integuments or the lips of human beings, as a cosmetic medium.

For the purposes of the invention, the expression “functionalized chain” means an alkyl chain comprising one or more functional or reactive groups chosen in particular from amide, hydroxyl, ether, oxyalkylene, polyoxyalkylene and halogen groups, including fluoro or perfluoro groups, ester, siloxane and polysiloxane groups. In addition, the hydrogen atoms of one or more fatty chains may be substituted at least partially with fluorine atoms.

According to the invention, these chains may be linked directly to the polymer skeleton or via an ester function or a perfluoro group.

For the purposes of the invention, the term “polymer” means a compound containing at least 2 repeating units and preferably at least 3 repeating units.

For the purposes of the invention, the expression “hydrocarbon-based repeating units” means a unit containing from 2 to 80 carbon atoms and preferably from 2 to 60 carbon atoms, bearing hydrogen atoms and optionally oxygen atoms, which may be linear, branched or cyclic, and saturated or unsaturated. These units each also comprise one or more hetero atoms that are advantageously non-pendent but are in the polymer skeleton. These hetero atoms are chosen from nitrogen, sulphur and phosphorus atoms and combinations thereof, optionally combined with one or more oxygen atoms. The units preferably comprise at least one nitrogen atom, in particular a non-pendent nitrogen atom. These units also advantageously comprise a carbonyl group, in particular when the hetero atom is a nitrogen atom in order to form an amide unit.

The units containing a hetero atom are, in particular, amide units forming a skeleton of the polyamide type, carbamate and/or urea units forming a polyurethane, polyurea and/or polyurea-urethane skeleton. These units are preferably amide units. The pendent chains are advantageously linked directly to at least one of the hetero atoms of the polymer skeleton. According to one embodiment, the first polymer comprises a polyamide skeleton.

Advantageously, the first polymer according to the invention is a nonionic polymer.

Between the hydrocarbon-based units, the first polymer may comprise silicone units or oxyalkylene units.

In addition, the first polymer in the composition of the invention advantageously comprises from 40% to 98% of fatty chains relative to the total number of units containing a hetero atom and of fatty chains, and better still from 50% to 95%. The nature and proportion of the units containing a hetero atom depends on the nature of the fatty phase and is, in particular, similar to the polar nature of the fatty phase. Thus, the more the units containing a hetero atom are polar and in high proportion in the first polymer, which corresponds to the presence of several hetero atoms, the greater the affinity of the first polymer for polar oils. On the other hand, the less polar or even apolar the units containing a hetero atom or the lower their proportion, the greater the affinity of the first polymer for apolar oils.

The first polymer is advantageously a polyamide. Thus, a subject of the invention is also a composition containing, in a cosmetically acceptable medium:

(i) a polyamide with a weight-average molecular mass of less than 100 000, comprising a) a polymer skeleton containing amide repeating units, and b) optionally at least one optionally functionalized pendent fatty chain and/or at least one optionally functionalized terminal chain, containing from 8 to 120 carbon atoms, which may be linked to these amide units,

(ii) an anionic polymer,

(iii) a cationic polymer.

The pendent fatty chains are preferably linked to at least one of the nitrogen atoms of the amide units of the first polymer.

In particular, the fatty chains of this polyamide represent from 40% to 98% of the total number of amide units and of fatty chains, and better still from 50% to 95%.

Advantageously, the first polymer, and in particular the polyamide, of the composition according to the invention has a weight-average molecular mass of less than 100 000 (especially ranging from 1 000 to 100 000), in particular less than 50 000 (especially ranging from 1 000 to 50 000) and more particularly ranging from 1 000 to 30 000, preferably from 2 000 to 20 000 and better still from 2 000 to 10 000.

The first polymer, and in particular the polyamide, is advantageously insoluble in water, especially at 25° C. In particular, it does not comprise any ionic groups.

As preferred first polymers which may be used in the invention, mention may be made of polyamides branched with pendent fatty chains and/or terminal fatty chains containing from 6 to 120 carbon atoms and better still from 8 to 120 and in particular from 12 to 68 carbon atoms, each terminal fatty chain being linked to the polyamide skeleton via at least one bonding group, in particular an ester. These polymers preferably comprise a fatty chain at each end of the polymer skeleton and in particular of the polyamide skeleton. Other bonding groups which may be mentioned are ether, amine, urea, urethane, thioester, thiourea and thiourethane groups.

These first polymers are preferably polymers resulting from a polycondensation between a dicarboxylic acid containing at least 32 carbon atoms (in particular containing from 32 to 44 carbon atoms) and an amine chosen from diamines containing at least 2 carbon atoms (in particular from 2 to 36 carbon atoms) and triamines containing at least 2 carbon atoms (in particular from 2 to 36 carbon atoms). The diacid is preferably a dimer of a fatty acid containing ethylenic unsaturation containing at least 16 carbon atoms, preferably from 16 to 24 carbon atoms, for instance oleic acid, linoleic acid or linolenic acid. The diamine is preferably ethylenediamine, hexylenediamine or hexamethylenediamine. The triamine is ethylene triamine for example. For the polymers comprising one or 2 terminal carboxylic acid groups, it is advantageous to esterify them with a monoalcohol containing at least 4 carbon atoms; preferably from 10 to 36 carbon atoms, better still from 12 to 24 and even better from 16 to 24, for example 18 carbon atoms.

These polymers are more especially those disclosed in document U.S. Pat. No. 5,783,657 from the company Union Camp. Each of these polymers in particular satisfies formula (I′) below:

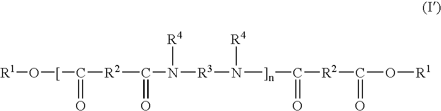

in which n denotes a number of amide units such that the number of ester groups represents from 10% to 50% of the total number of ester and amide groups; R¹ is, independently in each case, an alkyl or alkenyl group containing at least 4 carbon atoms and in particular from 4 to 24 carbon atoms; R² represents, independently in each case, a C₄ to C₄₂ hydrocarbon-based group, on condition that 50% of the groups R² represent a C₃₀ to C₄₂ hydrocarbon-based group; R³ represents, independently in each case, an organic group containing at least 2 carbon atoms, hydrogen atoms and optionally one or more oxygen or nitrogen atoms; and R⁴ represents, independently in each case, a hydrogen atom, a C₁ to C₁₀ alkyl group or a direct bond to R³ or to another R⁴₁, such that the nitrogen atom to which R³ and R⁴ are both attached forms part of a heterocyclic structure defined by R⁴—N—R³, with at least 50% of the groups R⁴ representing a hydrogen atom.

In the particular case of formula (I′), the terminal fatty chains that are optionally functionalized for the purposes of the invention are terminal chains linked to the last hetero atom, in this case nitrogen, of the polyamide skeleton.

In particular, the ester groups of formula (I′), which form part of the terminal and/or pendent fatty chains for the purposes of the invention, represent from 15% to 40% of the total number of ester and amide groups and better still from 20% to 35%. Furthermore, n is advantageously an integer ranging from 1 to 5 and better still greater than 2. Preferably, R¹ is a C₁₂ to C₂₂ and preferably c₁₆ to C₂₂ alkyl group. Advantageously, R² can be a C₁ to C₄₂ hydrocarbon-based (alkylene) group. Preferably, at least 50% and better still at least 75% of the groups R² are groups containing from 30 to 42 carbon atoms. The other groups R² are C₄ to C₁₉ and better still C₄ to C₁₂ hydrogen-containing groups. Preferably, R³ represents a C₂ to C₃₆ hydrocarbon-based group or a polyoxyalkylene group and R⁴ represents a hydrogen atom. Preferably, R³ represents a C₂ to C₁₂ hydrocarbon-based group.

The hydrocarbon-based groups may be linear, cyclic or branched, and saturated or unsaturated groups. Moreover, the alkyl and alkylene groups may be linear or branched, and saturated or unsaturated groups.

In general, the polymers of formula (I′) are in the form of mixtures of polymers, these mixtures also possibly containing a synthetic product corresponding to a compound of formula (I′) in which n is 0, i.e. a diester.

As examples of first polymers according to the invention, mention may be made of the commercial products sold by the company Arizona Chemical under the names Uniclear® 80 and Uniclear® 100. They are sold, respectively, in the form of an 80% (in terms of active material) gel in a mineral oil and a 100% (in terms of active material) gel. They have a softening point of from 88 to 94° C. These commercial products are a mixture of copolymers of a C₃₆ diacid condensed with ethylenediamine, having a weight-average molecular mass of about 6 000. The terminal ester groups result from the esterification of the remaining acid endings with cetyl alcohol, stearyl alcohol or mixtures thereof (also known as cetylstearyl alcohol).

As first polymers which can be used in the invention, mention may also be made of polyamide resins resulting from the condensation of an aliphatic dicarboxylic acid and a diamine (including compounds containing more than 2 carbonyl groups and 2 amine groups), the carbonyl and amine groups of adjacent individual units being condensed via an amide bond. These polyamide resins are, in particular, those sold under the brand name Versamid® by the companies General Mills Inc. and Henkel Corp. (Versamid®930,744 or 1655) or by the company Olin Mathieson Chemical Corp. under the brand name Onamid®, in particular Onamid® S or C. These resins have a weight-average molecular mass ranging from 6 000 to 9 000. For further information regarding these polyamides, reference may be made to the documents U.S. Pat. Nos. 3,645,705 and 3,148,125. More especially, Versamid® 930 or 744 is used.

The polyamides sold by the company Arizona Chemical under the references Uni-Rez® (2658, 2931, 2970, 2621, 2613, 2624, 2665, 1554, 2623 and 2662) and the product sold under the reference Macromelt 6212 by the company Henkel may also be used. For further information regarding these polyamides, reference may be made to document U.S. Pat. No. 5,500,209.

It is also possible to use polyamide resins obtained from plants, such as those disclosed in patents U.S. Pat. Nos. 5,783,657 and 5,998,570.

The first polymer present in the composition according to the invention advantageously has a softening point of greater than 65° C., which may be up to 190° C. It preferably has a softening point ranging from 70° C. to 130° C. and better still from 80° C. to 105° C. The first polymer is in particular a non-waxy polymer.

The first polymer according to the invention preferably corresponds to the formula (I) mentioned above. On account of its fatty chain(s), this first polymer is readily soluble in oils and thus leads to compositions that are macroscopically homogeneous even with a high content (at least 25%) of polymer, unlike polymers not containing a fatty chain.

The first polymer may be present in the composition according to the invention in a content ranging from 0.01% to 10% by weight, relative to the total weight of the composition, preferably ranging from 0.05% to 5% by weight and better still ranging from 0.1% to 3% by weight.

According to the invention, the composition according to the invention may contain any anionic film-forming polymer, which is preferably non-crosslinked, or cationic film-forming polymer that is known per se.

These polymers may be used in dissolved form or in the form of aqueous dispersions of solid polymer particles. The cationic polymer and the anionic polymer are different from the first polymer described above.

In the present patent application, the term “film-forming polymer” means a polymer that is capable, by itself or in the presence of an auxiliary film-forming agent, of forming a continuous film that adheres to a support, especially to keratin materials.

The anionic polymers generally used can be polymers comprising groups derived from carboxylic, sulphonic or phosphoric acid and can have a weight-average molecular weight of between about 500 and 5 000 000, and preferably greater than 100 000 and less than or equal to 5 000 000.

1) The carboxylic groups can be borne by unsaturated mono- or dicarboxylic acid monomers such as those corresponding to formula (I) below:

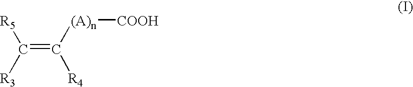

in which n is an integer from 0 to 10, A denotes a methylene group, optionally connected to the carbon atom of the unsaturated group or to the neighbouring methylene group when n is greater than 1 via a hetero atom such as oxygen or sulphur, R₅ denotes a hydrogen atom or a phenyl or benzyl group, R₃ denotes a hydrogen atom or a lower alkyl or carboxyl group, and R₄ denotes a hydrogen atom, a lower alkyl group or a —CH₂—COOH, phenyl or benzyl group.

In the abovementioned formula, the expression “lower alkyl radical” preferably denotes a group containing 1 to 4 carbon atoms and in particular methyl and ethyl.

The anionic polymers containing carboxylic groups which are preferred according to the invention are:

A) Homo- or copolymers of acrylic or methacrylic acid or salts thereof (in particular alkali metal, alkaline-earth metal or ammonium salts) and in particular the products sold under the names Versicol E or K by the company Allied Colloid, Ultrahold by the company BASF and Darvan 7 by the company Vanderbilt.

B) Copolymers of acrylic or methacrylic acids with a monoethylenic monomer such as ethylene, styrene, vinyl esters and acrylic or methacrylic acid esters. These copolymers can be grafted onto a polyalkylene glycol such as polyethylene glycol. Such polymers are described in particular in French patent 1 222 944 and German patent application 2 330 956. Mention may be made in particular of copolymers whose chain comprises an optionally N-alkylated and/or hydroxyalkylated acrylamide unit, such as those described in particular in the Luxembourg patent applications 75370 and 75371 or sold under the name Quadramer by the company American Cyanamid. Mention may also be made of the copolymers of acrylic acid and of acrylamide sold, in the form of their sodium salt, under the names Reten 421, 423 or 425 by the company Hercules. Mention may also be made of copolymers of acrylic acid and of C₁-C₄ alkyl methacrylate and terpolymers of vinylpyrrolidone, of (meth)acrylic acid and of (meth)acrylate of a C₁-C₂₀ alkyl, for example of lauryl (such as the product sold by the company ISP under the name Acrylidone LM), of tert-butyl (Luviflex VMB 70 sold by BASF) or of methyl (Stepanhold Extra sold by Stepan) and methacrylic acid/ethyl acrylate/tert-butyl acrylate terpolymers, such as the product sold under the name Luvimer 100 P by the company BASF.

C) Copolymers derived from crotonic acid, such as those whose chain comprises vinyl acetate or propionate units and optionally other monomers such as allylic or methallylic esters, vinyl ether or vinyl ester of a saturated, linear or branched carboxylic acid containing a long hydrocarbon-based chain such as those comprising at least 5 carbon atoms, it being possible for these polymers to be optionally grafted, or alternatively a vinyl, allylic or methallylic ester of an α- or β-cyclic carboxylic acid. Such polymers are described, inter alia, in French patents 1 222 944, 1 580 545, 2 265 782, 2 265 781, 1 564 110 and 2 439 798. Commercial products falling within this category are the resins 28-29-30, 26-13-14 and 28-13-10 sold by the company National Starch.

D) Copolymers derived from monounsaturated C₄-C₈ carboxylic acids or anhydrides chosen from:

copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and esters thereof, the anhydride functions of these copolymers optionally being monoesterified or monoamidated. Such polymers are described in particular in U.S. Pat. Nos. 2,047,398, 2,723,248 and 2,102,113 and GB patent 839 805, and in particular those sold under the names Gantrez AN or ES and Avantage CP by the company ISP.

Copolymers comprising (i) one or more maleic, citraconic or itaconic anhydrides and (ii) one or more monomers chosen from allylic or methallylic esters optionally comprising one or more acrylamide, methacrylamide, α-olefin, acrylic or methacrylic ester, acrylic or methacrylic acid or vinylpyrrolidone groups in their chain,

the anhydride functions of these copolymers optionally being monoesterified or monoamidated.

These polymers are described, for example, in French patents 2 350 384 and 2 357 241 by the Applicant.

E) Polyacrylamides comprising carboxylate groups,

F) the sodium salts of polyhydroxycarboxylic acids,

and mixtures thereof.

2) The polymers comprising sulphonic groups can be polymers comprising vinylsulphonic, styrenesulphonic, naphthalenesulphonic or acrylamidoalkylsulphonic units or alternatively sulphonic polyesters.

These polymers can be chosen in particular from:

polyvinylsulphonic acid salts with a weight-average molecular weight of between about 1000 and 100 000, as well as copolymers with an unsaturated comonomer such as acrylic or methacrylic acids and esters thereof, as well as acrylamide or derivatives thereof, vinyl ethers and vinylpyrrolidone;

polystyrenesulphonic acid salts, the sodium salts having a weight-average molecular weight of about 500 000 and of about 100 000, sold, respectively, under the names Flexan 500 and Flexan 130 by National Starch. These compounds are described in patent FR 2 198 719;

polyacrylamide sulphonic acid salts such as those mentioned in U.S. Pat. No. 4,128,631 and more particularly polyacrylamidoethylpropanesulphonic acid sold under the name Cosmedia Polymer HSP 1180 by Henkel;

sulphonic polyesters bearing at least one group —SO₃M with M representing a hydrogen atom, an ammonium ion NH₄⁺ or a metal ion. The copolyester can be, for example, a copolymer of at least one dicarboxylic acid, of at least one diol and of at least one difunctional aromatic monomer bearing a group —SO₃M with M representing a hydrogen atom, an ammonium ion NH₄⁺ or a metal ion.

The dicarboxylic acid can be chosen from phthalic acid, isophthalic acid and terephthalic acid. The diol can be chosen from ethylene glycol, diethylene glycol, triethylene glycol, 1,3-propanediol, 1,4-cyclohexanedimethanol and 1,4-butanediol. The difunctional aromatic monomer bearing the group —SO₃M can be chosen from sulphoisophthalic acid, in particular the sodium salt of 5-sulphoisophthalic acid, sulphoterephthalic acid, sulphophthalic acid and 4-sulphonaphthalene-2,7-dicarboxylic acid.

A preferred polyester which can be used is a polyester consisting essentially of repeating units of isophthalic acid, of diol and of sulphoisophthalic acid, and in particular the sulphopolyesters obtained by condensation of diethylene glycol, of cyclohexanedimethanol, of isophthalic acid and of sulphoisophthalic acid. Sulphonic polyesters which can be used are those sold under the names AQ55S, AQ38S and AQ29S by the company Eastman.

An anionic polymer which can also be used is (deoxy)ribonucleic acid.

According to the invention, the anionic polymers are preferably chosen from acrylic acid copolymers such as the acrylic acid/ethyl acrylate/N-tert-butylacrylamide terpolymers sold in particular under the name Ultrahold Strong by the company BASF, copolymers derived from crotonic acid, such as the vinyl acetate/vinyl tert-butylbenzoate/crotonic acid terpolymers and the crotonic acid/vinyl acetate/vinyl neododecanoate terpolymers sold in particular under the name Resin 28-29-30 by the company National Starch, polymers derived from maleic, fumaric or itaconic acids or anhydrides with vinyl esters, vinyl ethers, vinyl halides, phenylvinyl derivatives or acrylic acid and esters thereof, such as the methyl vinyl ether/monoesterified maleic anhydride copolymers sold, for example, under the name Gantrez by the company ISP, the copolymers of methacrylic acid and of methyl methacrylate sold under the name Eudragit L by the company Rohm Pharma, the methacrylic acid/methyl methacrylate/C1-C4 alkyl acrylate/acrylic acid or C1-C4 hydroxyalkyl methacrylate copolymers sold in the form of dispersions under the name Amerhold DR 25 by the company Amerchol or under the name Acudyne 255 by the company Rohm & Haas, the copolymers of methacrylic acid and of ethyl acrylate sold under the name Luvimer MAEX or MAE by the company BASF and the vinyl acetate/crotonic acid copolymers and vinyl acetate/crotonic acid copolymers grafted with polyethylene glycol sold under the name Aristoflex A by the company BASF, the acrylic or methacrylic acid homopolymers sold, for example, under the name Versicol E 5 or poly(sodium methacrylate) sold under the name Darvan 7 by the company Vanderbilt, and mixtures thereof.

The anionic polymers which are more particularly preferred are chosen from non-crosslinked anionic polymers such as the methyl vinyl ether/monoesterified maleic anhydride copolymers sold under the name Gantrez ES 425 by the company ISP, the acrylic acid/ethyl acrylate/N-tert-butylacrylamide terpolymers sold under the name Ultrahold Strong by the company BASF, the copolymers of methacrylic acid and of methyl methacrylate sold under the name Eudragit L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name Luvimer MAEX or MAE by the company BASF, the vinylpyrrolidone/acrylic acid/lauryl methacrylate terpolymers sold under the name Acrylidone LM by the company ISP and the acrylic or methacrylic acid homopolymers sold, for example, under the name Versicol E 5 or poly(sodium methacrylate) sold under the name Darvan 7 by the company Vanderbilt, and mixtures thereof.

According to the invention, it is also possible to use anionic polymers in latex or pseudolatex form, i.e. in the form of a dispersion of insoluble polymer particles.

3) According to the invention, it is also possible to use anionic polymers of grafted silicone type comprising a polysiloxane portion and a portion consisting of a non-silicone organic chain, one of the two portions constituting the main chain of the polymer, the other being grafted onto the said main chain. These polymers are described, for example, in patent applications EP-A-0 412 704, EP-A-0 412 707, EP-A-0 640 105 and WO 95/00578, EP-A-0 582 152 and WO 93/23009 and U.S. Pat. Nos. 4,693,935, 4,728,571 and 4,972,037.

Such polymers are, for example, the copolymers which can be obtained by radical polymerization from a monomer mixture consisting of:

a) 50 to 90% by weight of tert-butyl acrylate;

b) 1 to 40% by weight of acrylic acid;

c) 5 to 40% by weight of silicone macromer of formula (II):

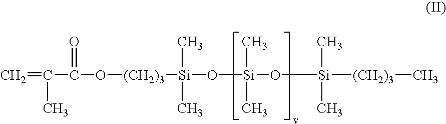

with v being a number ranging from 5 to 700; the weight percentages being calculated relative to the total weight of the monomers.

One family of silicone polymers containing a polysiloxane skeleton grafted with non-silicone organic monomers which is particularly suitable for carrying out the present invention consists of silicone polymers whose structure comprises the unit of formula (III) below:

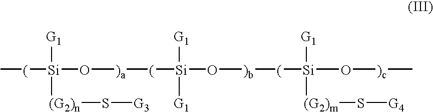

in which the radicals G₁, which may be identical or different, represent hydrogen or a C₁-C₁₀ alkyl radical or alternatively a phenyl radical; the radicals G₂, which may be identical or different, represent a C₁-C₁₀ alkylene group; G₃ represents a polymer residue resulting from the (homo)polymerization of at least one ethylenically unsaturated anionic monomer; G₄ represents a polymer residue resulting from the (homo)polymerization of at least one ethylenically unsaturated hydrophobic monomer; m and n are equal to 0 or 1; a is an integer ranging from 0 to 50; b is an integer which can be between 10 and 350, c is an integer ranging from 0 to 50; with the proviso that one of the parameters a and c is other than 0.

Preferably, the unit of formula (III) above has at least one, and even more preferably all, of the following characteristics:

the radicals G₁ denote a C₁-C₁₀ alkyl radical, preferably a methyl radical;

n is non-zero and the radicals G₂ represent a divalent C₁-C₃ radical, preferably a propylene radical;

G₃ represents a polymer radical resulting from the (homo)polymerization of at least one monomer such as an ethylenically unsaturated carboxylic acid, preferably acrylic acid and/or methacrylic acid;

G₄ represents a polymer radical resulting from the (homo)polymerization of at least one monomer such as a C₁-C₁₀ alkyl (meth)acrylate, preferably isobutyl or methyl (meth)acrylate.

Preferably, the unit of formula (III) above can also have all of the following characteristics:

the radicals G₁ denote an alkyl radical, preferably a methyl radical;

n is non-zero and the radicals G₂ represent a divalent C₁-C₃ radical, preferably a propylene radical;

G₃ represents a polymer radical resulting from the (homo)polymerization of at least one monomer such as an ethylenically unsaturated carboxylic acid, preferably acrylic acid and/or methacrylic acid;

c is equal to zero.

Examples of grafted silicone polymers are, in particular, polydimethylsiloxanes (PDMSs) onto which are grafted, via a connecting member of thiopropylene type, mixed polymer units of the poly(meth)acrylic acid type and of the poly(alkyl (meth)acrylate) type, such as poly(isobutyl (meth)acrylate).

The grafted silicone polymers of formula (III) of polymethyl/methylsiloxane structure containing 3-thiopropyl polymethacrylic acid groups and 3-thiopropyl polymethyl methacrylate groups and the grafted silicone polymers of formula (III) of polymethyl/methylsiloxane structure containing 3-thiopropyl polyacrylic acid groups are particularly used.

According to the invention, the anionic polymer(s) can be present in a content ranging from 0.01% to 20% by weight, preferably from 0.05% to 15% by weight, and even more preferably from 0.1% to 7% by weight, relative to the total weight of the composition.

The cationic polymers which can be used in accordance with the present invention can be chosen from all those already known per se, and in particular from those described in patent application EP-A-0 337 354 and in French patent applications FR-A-2 270 846, 2 383 660, 2 598 611, 2 470 596 and 2 519 863.

Even more generally, for the purposes of the present invention, the expression “cationic polymer” denotes any polymer containing cationic groups or groups which can be ionized into cationic groups.

The preferred cationic polymers are chosen from those which contain units comprising primary, secondary, tertiary and/or quaternary amine groups which can either form part of the main polymer chain or can be borne by a lateral substituent directly connected thereto.

The cationic polymers used generally have a number-average molecular mass of between 500 and 5×10⁶ approximately, preferably between 10³ and 3×10⁶ approximately and better still greater than 100 000 and less than or equal to 3×10⁶.

Among the cationic polymers that may be mentioned more particularly are polymers such as polyamines, polyaminoamides and polyquaternary ammoniums. These are known products.

One family of cationic polymers is the family of silicone cationic polymers. Among these polymers which may be mentioned are:

(a) the silicone polymers corresponding to formula (IV) below:

R⁶ₐG⁵₃₋ₐ—Si (OSiG⁶₂)ₙ—(OSiG⁷_{b}R⁷_{2-b})ₘ—O—SiG⁸_{3-a′}, —R⁸_{a′}, (IV)

in which:

G⁵, G⁶, G⁷ and G⁸, which may be identical or different, denote a hydrogen atom, a phenyl or OH group, a C₁-C₁₈ alkyl group, for example methyl, a C₂-C₁₈ alkenyl group or a C₁-C₁₈ alkoxy group, a and a′, which may be identical or different, denote the number 0 or an integer from 1 to 3, in particular 0,

b denotes 0 or 1, and in particular 1,

m and n are numbers such that the sum (n+m) can range especially from 1 to 2000 and in particular from 50 to 150, it being possible for n to denote a number from 0 to 1999 and in particular from 49 to 149, and for m to denote a number from 1 to 2000 and in particular from 1 to 10;

R⁶, R⁷ and R⁸, which may be identical or different, denote a monovalent radical of formula —C_{q}H_{2q}OₛR⁹ₜL in which q is a number from 1 to 8, s and t, which may be identical or different, are equal to 0 or 1, R⁹ denotes an optionally hydroxylated alkylene group and L is an optionally quaternized amino group chosen from the groups:

—NR″—CH₂—CH₂—N′ (R″)₂

—N(R″)₂

—N^{⊕}(R″)₃A⁻

—N^{⊕}H(R″)₂A⁻

—N^{⊕}H₂ (R″)A⁻

—N(R″)—CH₂—CH₂—N^{⊕}R″H₂A⁻,

in which R″ can denote hydrogen, phenyl, benzyl or a monovalent saturated hydrocarbon-based radical, for example an alkyl radical containing from 1 to 20 carbon atoms, and A⁻ represents a halide ion such as, for example, fluoride, chloride, bromide or iodide.

Products corresponding to this definition are, for example, the polysiloxanes referred to in the CTFA dictionary as “amodimethicone” and corresponding to formula (V) below:

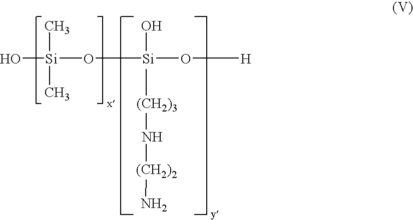

in which x′ and y′ are integers dependent on the molecular weight, generally such that the said molecular weight is between 5000 and 20 000 approximately.

One product corresponding to formula (IV) is the polymer referred to in the CTFA dictionary as “trimethylsilylamodimethicone”, corresponding to formula (VI):

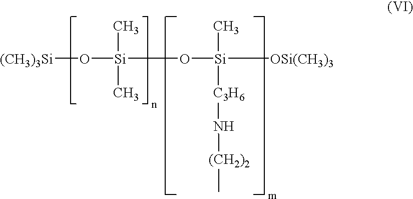

in which n and m have the meanings given above for formula (IV).

A commercial product corresponding to this definition is a mixture (90/10 by weight) of a polydimethylsiloxane containing aminoethyl aminoisobutyl groups and of a polydimethylsiloxane sold under the name Q2-8220 by the company Dow Corning.

Such polymers are described, for example, in patent application EP-A-95238.

Other polymers corresponding to formula (IV) are the silicone polymers corresponding to formula (VII) below:

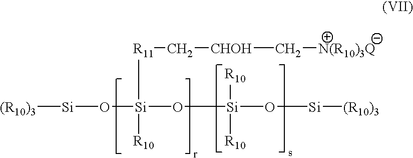

in which:

R₁₀ represents a monovalent hydrocarbon-based radical containing from 1 to 18 carbon atoms, and in particular a C₁-C₁₈ alkyl or C₂-C₁₈ alkenyl radical, for example methyl;

R₁₁ represents a divalent hydrocarbon-based radical, in particular a C₁-C₈ alkylene radical or a divalent C₁-C₁₈, for example C₁-C₈, alkylenoxy radical;

Q⁻ is halide ion, in particular chloride;

r represents an average statistical value from 2 to 20 and in particular from 2 to 8;

s represents an average statistical value from 20 to 200 and in particular from 20 to 50.

Such polymers are described more particularly in U.S. Pat. No. 4,185,087.

(b) the compounds of formula: NH—[(CH₂)₃—Si[OSi(CH₃)₃]]₃ corresponding to the CTFA name “aminobispropyl-dimethicone”.

One polymer falling within this category is the polymer sold by the company Union Carbide under the name “Ucar Silicone ALE 56”.

When these silicone polymers are used, one particularly advantageous embodiment is their joint use with cationic and/or nonionic surfactants. It is possible, for example, to use the product sold under the name “Cationic Emulsion DC 929” by the company Dow Corning, which comprises, besides amodimethicone, a cationic surfactant, comprising a mixture of products corresponding to formula (VIII):

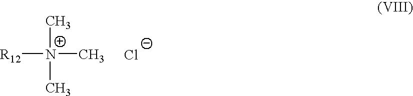

in which R₁₂ denotes alkenyl and/or alkyl radicals containing from 14 to 22 carbon atoms, derived from tallow fatty acids, in combination with a nonionic surfactant of formula:

C₉H₁₉—C₆H₄—(OC₂H₄)₁₀—OH

known under the name “Nonoxynol 10”.

Another commercial product which can be used according to the invention is the product sold under the name “Dow Corning Q2 7224” by the company Dow Corning comprising, in combination, trimethylsilylamodimethicone of formula (IV), a nonionic surfactant of formula: C₈H₁₇—C₆H₄—(OCH₂CH₂)ₙ—OH in which n=40, also known as octoxynol-40, another nonionic surfactant of formula: C₁₂H₂₅—(OCH₂—CH₂)ₙ—OH in which n=6, also known as isolaureth-6, and glycol.

The polymers of polyamine, polyaminoamide and polyquaternary ammonium type which can be used in accordance with the present invention and which can be mentioned in particular are those described in French patents No. 2 505 348 or 2 542 997. Among these polymers which may be mentioned are:

(1) Quaternized or non-quaternized vinylpyrrolidone/dialkylaminoalkyl acrylate or methacrylate copolymers, such as the products sold under the name “Gafquat®” by the company ISP, such as, for example, Gafquat 734, 755 or HS100, or alternatively the product known as “Copolymer 937”. These polymers are described in detail in French patents 2 077 143 and 2 393 573.

(2) Cellulose ether derivatives, in particular hydroxy(C1-C4)alkylcelluloses, comprising quaternary ammonium groups described in French patent 1 492 597, and in particular the polymers sold under the names “JR” (JR 400, JR 125, JR 30M) or “LR” (LR 400, LR 30M) by the company Union Carbide Corporation. These polymers are also defined in the CTFA dictionary as quaternary ammoniums of hydroxyethylcellulose which has reacted with an epoxide (in particular epichlorohydrin) substituted with a trimethylammonium group.

(3) Cationic cellulose derivatives such as cellulose copolymers or cellulose derivatives grafted with a water-soluble quaternary ammonium monomer, and described in particular in U.S. Pat. No. 4,131,576, such as hydroxyalkylcelluloses, for example hydroxymethyl-, hydroxyethyl- or hydroxypropylcelluloses grafted in particular with a methacryloylethyltrimethylammonium, methacrylamidopropyltrimethylammonium or dimethyldiallylammonium salt.

The commercial products corresponding to this definition are, more particularly, the products sold under the names “Celquat L 200” and “Celquat H 100” by the company National Starch.

(4) The cationic polysaccharides described more particularly in U.S. Pat. Nos. 3,589,578 and 4,031,307 and more particularly the product sold under the name “Jaguar C.13 S” sold by the company Meyhall.

(5) Polymers consisting of piperazinyl units and of divalent alkylene or hydroxyalkylene radicals containing straight or branched chains, optionally interrupted with oxygen, sulphur or nitrogen atoms or with aromatic or heterocyclic rings, as well as the oxidation and/or quaternization products of these polymers. Such polymers are described in particular in French patents 2 162 025 and 2 280 361.

(6) Water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or with an oligomer resulting from the reaction of a difunctional compound which is reactive with respect to a bis-halohydrin, a bis-azetidinium, a bis-haloacyldiamine, a bis-alkyl halide, an epihalohydrin, a diepoxide or a bis-unsaturated derivative, the crosslinking agent being used in proportions ranging from 0.025 to 0.35 mol per amine group of the polyaminoamide; these polyaminoamides can be alkylated or, if they comprise one or more tertiary amine functions, can be quaternized. Such polymers are described in particular in French patents 2 252 840 and 2 368 508.

(7) Polyaminoamide derivatives resulting from the condensation of polyalkylene polyamines with polycarboxylic acids, followed by an alkylation with difunctional agents. Mention may be made, for example, of adipic acid/dialkylaminohydroxyalkyldialkylenetri-amine polymers in which the alkyl radical comprises from 1 to 4 carbon atoms and preferably denotes methyl, ethyl or propyl. Such polymers are described in particular in French patent 1 583 363.

Among these derivatives which may be mentioned more particularly are the adipic acid/dimethylaminohydroxypropyl/diethylenetriamine polymers sold under the name “Cartaretine F, F4 or F8” by the company Sandoz.

(8) Polymers obtained by reacting a polyalkylene polyamine comprising two primary amine groups and at least one secondary amine group with a dicarboxylic acid chosen from diglycolic acid and saturated aliphatic dicarboxylic acids containing from 3 to 8 carbon atoms. The molar ratio between the polyalkylene polyamine and the dicarboxylic acid is between 0.8:1 and 1.4:1, the polyaminoamide resulting therefrom being made to react with epichlorohydrin in a molar ratio of epichlorohydrin relative to the secondary amine group in the polyaminoamide of between 0.5:1 and 1.8:1. Such polymers are described in particular in U.S. Pat. Nos. 3,227,615 and 2,961,347.

Polymers of this type are sold in particular under the name “Hercosett 57” by the company Hercules Inc. or alternatively under the name “PD 170” or “Delsette 101” by the company Hercules in the case of the adipic acid/epoxypropyl/diethylenetriamine copolymer.

(9) Copolymers of methyldiallylamine or of diallyldimethylammonium, such as homopolymers or copolymers comprising, as main constituent of the chain, units corresponding to formula (IX) or (IX′):

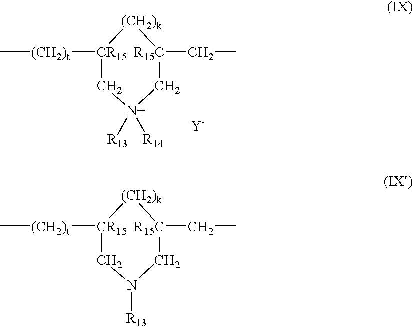

in which formulae k and t are equal to 0 or 1, the sum k+t being equal to 1; R₁₅ denotes a hydrogen atom or a methyl radical; R₁₃ and R₁₄, independently of each other, denote an alkyl group containing from 1 to 22 carbon atoms, a hydroxyalkyl group in which the alkyl group preferably contains 1 to 5 carbon atoms, or a lower amidoalkyl group, or R₁₃ and R₁₄ can denote, together with the nitrogen atom to which they are attached, heterocyclic groups such as piperidyl or morpholinyl; Y⁻ is an anion such as bromide, chloride, acetate, borate, citrate, tartrate, bisulphate, bisulphite, sulphate or phosphate. These polymers are described in particular in French patent 2 080 759 and in its certificate of addition 2 190 406.

Mention may be made, for example, of the diallyldimethylammonium chloride homopolymer sold under the name “Merquat 100” by the company Merck and the copolymers of diallyldimethylammonium chloride and of acrylamide sold under the name “Merquat 550”.

(10) The diquaternary ammonium polymer containing repeating units corresponding to formula (X):

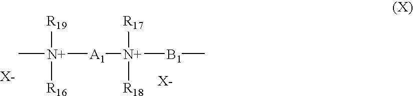

in which formula (X):

R₁₆, R₁₇, R₁₈ and R₁₉, which may be identical or different, represent aliphatic, alicyclic or arylaliphatic radicals containing from 1 to 20 carbon atoms or lower hydroxyalkyl aliphatic radicals, or R₁₆, R₁₇, R₁₈ and R₁₉, together or separately, constitute, with the nitrogen atoms to which they are attached, heterocycles optionally containing a second hetero atom other than nitrogen, or alternatively R₁₆, R₁₇, R₁₈ and R₁₉ represent a linear or branched C₁-C₆ alkyl radical substituted with a nitrile, ester, acyl, amide or —CO—O—R₂₀—D or —CO—NH—R₂₀—D group in which R₂₀ is an alkylene and D is a quaternary ammonium group;

A₁ and B₁ represent polymethylenic groups containing from 2 to 20 carbon atoms which can be linear or branched, saturated or unsaturated and which can contain, linked to or intercalated in the main chain, one or more aromatic rings, one or more oxygen or sulphur atoms or sulphoxide, sulphone, disulphide, amino, alkylamino, hydroxyl, quaternary ammonium, ureido, amide or ester groups, and

X⁻ denotes an anion derived from an inorganic or organic acid;

Aₗ, R₁₆ and R₁₈ can form, with the two nitrogen atoms to which they are attached, a piperazine ring; in addition, if Al denotes a linear or branched, saturated or unsaturated alkylene or hydroxyalkylene radical, B₁ can also denote a group (CH2)ₙ—CO—D—OC—(CH2)ₙ—

in which n denotes an integer ranging from 1 to 6 and D denotes:

a) a glycol residue of formula: —O—Z—O—, in which Z denotes a linear or branched hydrocarbon-based radical or a group corresponding to one of the following formulae:

—(CH₂—CH₂—O)ₓ—CH₂—CH₂

—[CH₂—CH(CH₃)—O]_{y}—CH₂—CH(CH₃)—

in which x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH— in which Y denotes a linear or branched hydrocarbon-based radical, or alternatively the divalent radical

—CH₂—CH₂—S—S—CH₂—CH₂—;

d) a ureylene group of formula: —NH—CO—NH—.

Preferably, X⁻ is an anion such as chloride or bromide.

These polymers have a number-average molecular mass generally of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

(11) Polyquaternary ammonium polymers consisting of units of formula (XI):

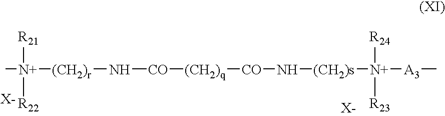

in which formula:

R₂₁, R₂₂, R₂₃ and R₂₄, which may be identical or different, represent a hydrogen atom or a methyl, ethyl, propyl, β-hydroxyethyl, β-hydroxypropyl or —CH₂CH₂ (OCH₂CH₂)ₚOH radical,

in which p is equal to 0 or to an integer between 1 and 6, with the proviso that R₂₁, R₂₂, R₂₃ and R₂₄ do not simultaneously represent a hydrogen atom,

r and s, which may be identical or different, are integers between 1 and 6,

q is equal to 0 or to an integer between 1 and 34,

X denotes a halogen atom,

A₃ denotes a dihalide radical or preferably represents —CH₂—CH₂—O—CH₂—CH₂—.

Such compounds are described in particular in patent application EP-A-122 324.

Among the products which may be mentioned, for example, are “Mirapol® A 15”, “Mirapol® AD1”, “Mirapol® AZ1” and “Mirapol® 175” sold by the company Miranol.

(12) Homopolymers or copolymers derived from acrylic or methacrylic acids and comprising units of formulae (XII), (XIII) and (XIV) below:

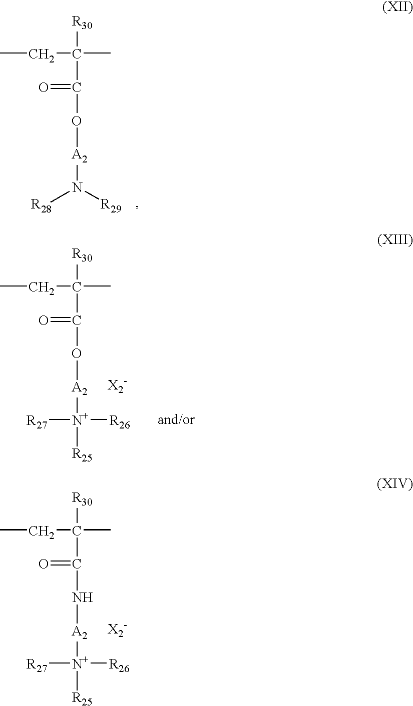

in which the groups R₃₀ independently denote H or CH₃,

the groups A₂ independently denote a linear or branched alkyl group of 1 to 6 carbon atoms or a hydroxyalkyl group of 1 to 4 carbon atoms,

the groups R₂₅, R₂₆ and R₂₇, which may be identical or different, independently denote an alkyl group of 1 to 18 carbon atoms or a benzyl radical,

the groups R₂₈ and R₂₉ represent a hydrogen atom or an alkyl group of 1 to 6 carbon atoms,

X₂⁻ denotes an anion, for example methosulphate or halide, such as chloride or bromide.

The comonomer(s) which can be used to prepare the corresponding copolymers belong to the family of acrylamides, methacrylamides, diacetbneacrylamides and acrylamides and methacrylamides substituted on the nitrogen with lower alkyls, alkyl esters, acrylic or methacrylic acids, vinylpyrrolidone or vinyl esters.

(13) Quaternary vinylpyrrolidone and vinylimidazole polymers such as, for example, the products sold under the names Luviquat® FC 905, FC 550 and FC 370 by the company BASF.

(14) Polyamines such as Polyquart H sold by Henkel, referred to under the name “Polyethylene glycol (15) tallow polyamine” in the CTFA dictionary.

(15) Crosslinked methacryloyloxyethyltrimethylammonium chloride polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide. An acrylamide/methacryloyloxyethyltrimethylammonium chloride crosslinked copolymer (20/80 by weight) in the form of a dispersion containing 50% by weight of the said copolymer in mineral oil can be used more particularly. This dispersion is sold under the name “Salcare SC 92” by the company Allied Colloids. A methacryloyloxyethyltrimethylammonium chloride crosslinked homopolymer containing about 50% by weight of the homopolymer in mineral oil can also be used. This dispersion is sold under the name “Salcare® SC 95” by the company Allied Colloids.

Other cationic polymers which can be used in the context of the invention are polyalkyleneimines, in particular polyethyleneimines, polymers containing vinylpyridine or vinylpyridinium units, condensates of polyamines and of epichlorohydrin, polyquaternary ureylenes and chitin derivatives.

Among all the cationic polymers which can be used in the context of the present invention, it is preferred to use cyclopolymers, in particular the copolymers of dimethyldiallylammonium chloride and of acrylamide with a molecular weight greater than 500 000, sold under the names “Merquat® 550” and “Merquat® S” by the company Merck, cationic polysaccharides and more particularly the polymer sold under the name “Jaguar® C13S” by the company Meyhall, and the polyaminoamides of the family (6) described above.

According to the invention, cationic polymers in the form of a latex or a pseudolatex, i.e. in the form of a dispersion of insoluble polymer particles, can also be used.

According to the invention, the cationic polymer(s) can be present in a content ranging from 0.01% to 20% by weight, preferably from 0.01% to 15% by weight and even more preferably from 0.05% to 5% by weight, relative to the total weight of the composition.

The cationic charge of the cationic polymer(s)/anionic charge of the anionic polymer(s) ratio, expressed in meq./g, is generally between 0.25 and 5, preferably between 0.5 and 2 and even more preferably between 0.75 and 1.25.

The cationic charge is the number of quaternary, tertiary, secondary or primary amine atoms per gram of polymer.

The cationic polymer can advantageously be a hydroxy(C₁-C₄)alkylcellulose comprising quaternary ammonium groups, in particular a hydroxyethylcellulose crosslinked with epichlorohydrin quaternized with trimethylamine; the anionic polymer can be a poly(sodium methacrylate).

The fatty phase of the composition can comprise fatty substances chosen from oils, organic solvents, waxes and pasty fatty substances, and mixtures thereof. The fatty phase can form a continuous phase of the composition.

The fatty phase may especially consist of any oil which is physiologically acceptable and in particular cosmetically acceptable, chosen especially from carbon-based oils, hydrocarbon-based oils, fluoro oils and/or silicone oils of mineral, animal, plant or synthetic origin, alone or as a mixture, provided that they form a homogeneous and stable mixture and provided that they are compatible with the intended use.

The total fatty phase of the composition can represent from 1% to 99% by weight, relative to the total weight of the composition, and preferably from 5% to 85% by weight.

The fatty phase of the composition can advantageously comprise at least one volatile oil or organic solvent and/or at least one non-volatile oil.

For the purposes of the invention, the expression “volatile oil or organic solvent” means any non-aqueous medium which can evaporate on contact with the skin in less than one hour at room temperature and atmospheric pressure. The volatile organic solvent(s) and the volatile oils of the invention are volatile cosmetic organic solvents and oils, that are liquid at room temperature, having a non-zero vapour pressure at room temperature and atmospheric pressure, ranging in particular from 10⁻² to 300 mmHg (0.13 Pa to 40 000 Pa) and preferably greater than 0.3 mmHg (30 Pa). The expression “non-volatile oil” means an oil which remains on the skin at room temperature and atmospheric pressure for at least several hours and which in particular has a vapour pressure of less than 10⁻² mmHg (1.33 Pa).

These oils may be hydrocarbon-based oils, silicone oils or fluoro oils, or mixtures thereof.

The expression “hydrocarbon-based oil” means an oil mainly containing hydrogen and carbon atoms and optionally oxygen, nitrogen, sulphur or phosphorus atoms. The volatile hydrocarbon-based oils may be chosen from hydrocarbon-based oils containing from 8 to 16 carbon atoms, and especially C₈-C₁₆ branched alkanes, for instance C₈-C₁₆ isoalkanes of petroleum origin (also known as isoparaffins), for instance isododecane (also known as 2,2,4,4,6-pentamethylheptane), isodecane and isohexadecane, and, for example, the oils sold under the trade names Isopars or Permetyls, C₈-C₁₆ branched esters, isohexyl neopentanoate, and mixtures thereof. Other volatile hydrocarbon-based oils, for instance petroleum distillates, especially those sold under the name Shell Solt by the company Shell, may also be used. The volatile solvent is preferably chosen from hydrocarbon-based volatile oils containing from 8 to 16 carbon atoms, and mixtures thereof.

Volatile oils which may also be used are volatile silicones such as, for example, linear or cyclic volatile silicone oils, especially those with a viscosity ≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils which may be used in the invention, mention may be made in particular of octamethylcyclotetrasiloxane, decamethylcyclopentasiloxane, dodecamethyl-cyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Volatile fluoro solvents such as nonafluoromethoxybutane or perfluoromethylcyclopentane may also be used.

The volatile oil may be present in the composition according to the invention in a content ranging from 0% to 98% by weight (in particular from 0.1% to 98%), relative to the total weight of the composition, preferably from 0% to 65% by weight (in particular from 1% to 65%).

The composition can also comprise at least one non-volatile oil chosen in particular from non-volatile hydrocarbon-based and/or silicone and/or fluoro oils.

Non-volatile hydrocarbon-based oils which may be mentioned in particular are:

hydrocarbon-based plant oils such as triglycerides consisting of fatty acid esters and of glycerol in which the fatty acids may have varied chain lengths from C₄ to C₂₄, these chains possibly being linear or branched, and saturated or unsaturated; these oils are, in particular, wheat germ oil, sunflower oil, grape seed oil, sesame oil, corn oil, apricot oil, castor oil, karite butter, avocado oil, olive oil, soybean oil, sweet almond oil, palm oil, rape seed oil, cotton oil, hazelnut oil, macadamia oil, jojoba oil, alfalfa oil, poppy oil, pumpkin oil, marrow oil, blackcurrant seed oil, evening primrose oil, millet oil, barley oil, quinoa oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel;

synthetic ethers containing from 10 to 40 carbon atoms;

linear or branched hydrocarbons of mineral or synthetic origin, such as petroleum jelly, polydecenes, hydrogenated polyisobutene such as parleam, and squalane, and mixtures thereof;

synthetic esters such as oils of formula R₁COOR₂ in which R₁ represents a linear or branched fatty acid residue containing from 1 to 40 carbon atoms and R₂ represents an in particular branched hydrocarbon-based chain containing from 1 to 40 carbon atoms, on condition that R₅+R₆∃10, such as, for example, purcellin oil (cetostearyl octanoate), isopropyl myristate, isopropyl palmitate, C₁₂-C₁₅ alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearyl lactate and diisostearyl malate; and pentaerythritol esters;

fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;

higher fatty acids such as oleic acid, linoleic acid or linolenic acid;

and mixtures thereof.

The non-volatile silicone oils which may be used in the composition according to the invention may be non-volatile polydimethylsiloxanes (PDMSs), polydimethylsiloxanes comprising alkyl or alkoxy groups, that are pendent and/or at the end of a silicone chain, the groups each containing from 2 to 24 carbon atoms, phenylsilicones, for instance phenyltrimethicones, phenyldimethicones, phenyl-trimethylsiloxydiphenylsiloxanes, diphenyldimethicones, diphenylmethyldiphenyltrisiloxanes and 2-phenylethyl trimethylsiloxysilicates.

The fluoro oils which may be used in the invention are, in particular, fluorosilicone oils, fluoropolyethers or fluorosilicones, as described in document EP-A-847 752.

The non-volatile oils may be present in the composition according to the invention in a content ranging from 0% to 80% (in particular from 0.1% to 80%) by weight, preferably from 0% to 50% by weight (in particular 0.1% to 50% by weight), relative to the total weight of the composition, and better still from 0% to 20% by weight (in particular 0.1% to 20%).

The fatty phase of the composition according to the invention can comprise a wax. For the purposes of the present invention, the term “wax” means a lipophilic fatty compound that is solid at room temperature (25° C.) and atmospheric pressure (760 mmHg, i.e. 10⁵ Pa), which undergoes a reversible solid/liquid change of state and which has a melting point of greater than. 30° C. and better still greater than 55° C., which may be up to 200° C., in particular up to 120° C.

By taking the wax to its melting point, it is possible to make it miscible with oils and to form a microscopically homogeneous mixture, but on returning the temperature of the mixture to room temperature, recrystallization of the wax in the mixture of oils is obtained.

According to the invention, the melting point values correspond to the melting peak measured using a differential scanning calorimeter (DSC), for example the calorimeter sold under the name DSC 30 by the company Mettler, with a temperature increase of 5 or 10° C. per minute.

For the purposes of the invention, the waxes are those generally used in cosmetics and dermatology. Mention may be made in particular of beeswax, lanolin wax, Chinese insect waxes, rice wax, carnauba wax, candelilla wax, ouricury wax, sugar cane wax, Japan wax, sumach wax, montan wax, microcrystalline waxes, paraffin waxes, ozokerites, ceresin wax, lignite wax, polyethylene waxes and the waxes obtained by Fisher-Tropsch synthesis, and fatty acid esters of glycerides that are solid at 40° C. and better still at more than 55° C.

Mention may also be made of the waxes obtained by catalytic hydrogenation of animal or plant oils containing linear or branched C₈-C₃₂ fatty chains. Among these, mention may be made in particular of hydrogenated jojoba oil, hydrogenated sunflower oil, hydrogenated castor oil, hydrogenated coconut oil and hydrogenated lanolin oil.

Mention may also be made of silicone waxes or fluoro waxes.

The waxes present in the composition may be dispersed in the form of particles in an aqueous medium. These particles may have an average size ranging from 50 μm to 10 μm and preferably from 50 μm to 3.5 μm.

In particular, the wax may be present in the form of a wax-in-water emulsion, the waxes possibly being in the form of particles with an average size ranging from 1 μm to 10 μm and preferably from 1 μm to 3.5 μm.

In another embodiment of the composition according to the invention, the wax may be present in the form of a wax microdispersion, the wax being in the form of particles with an average size of less than 1 μm and in particular ranging from 50 μm to 500 μm . Wax microdispersions are disclosed in documents EP-A-557 196 and EP-A-1 048 282.

The wax may also have a hardness ranging from 0.05 MPa to 15 MPa and preferably ranging from 6 MPa to 15 MPa. The hardness is determined by measuring the compressive strength, measured at 20° C. using a texturometer sold under the name TA-XT2i by the company Rheo, equipped with a stainless steel cylinder 2 mm in diameter travelling at a measuring speed of 0.1 mm/s, and penetrating into the wax to a penetration depth of 0.3 mm. To carry out the hardness measurement, the wax is melted at a temperature equal to the melting point of the wax +20° C. The molten wax is cast in a container 30 mm in diameter and 20 mm deep. The wax is recrystallized at room temperature (25° C.) over 24 hours and is then stored for at least one hour at 20° C. before carrying out the hardness measurement. The value of the hardness is the compressive strength measured divided by the area of the texturometer cylinder in contact with the wax.

The wax may be present in the composition according to the invention in a content ranging from 0.1% to 50% by weight, relative to the total weight of the composition, preferably from 0.5% to 40% by weight and better still from 1% to 30% by weight.

The composition according to the invention may contain at least one fatty compound that is pasty at room temperature. For the purposes of the invention, the expression “pasty fatty substance” means fatty substances with a melting point ranging from 20 to 55° C., preferably 25 to 45° C., and/or a viscosity at 40° C. ranging from 0.1 to 40 Pa.s (1 to 400 poises), preferably 0.5 to 25 Pa.s, measured using a Contraves TV or Rheomat 80 viscometer, equipped with a spindle rotating at 60 Hz. A person skilled in the art can select the spindle for measuring the viscosity from the spindles MS-r3 and MS-r4, on the basis of his general knowledge, so as to be able to carry out the measurement of the pasty compound tested.

These fatty substances are preferably hydrocarbon-based compounds, optionally of polymeric type; they can also be chosen from silicone compounds and/or fluoro compounds; they may also be in the form of a mixture of hydrocarbon-based compounds and/or silicone compounds and/or fluoro compounds. In the case of a mixture of different pasty fatty substances, the hydrocarbon-based pasty compounds (containing mainly hydrogen and carbon atoms and optionally ester groups) are preferably used in major proportion.

Among the pasty compounds which may be used in the composition according to the invention, mention may be made of lanolins and lanolin derivatives such as acetylated lanolins or oxypropylenated lanolins or isopropyl lanolate, having a viscosity of from 18 to 21 Pa.s, preferably 19 to 20.5 Pa.s, and/or a melting point of from 30 to 55° C., and mixtures thereof. It is also possible to use esters of fatty acids or of fatty alcohols, in particular those containing from 20 to 65 carbon atoms (melting point of about from 20 to 35° C. and/or viscosity at 40° C. ranging from 0.1 to 40 Pa.s), such as triisostearyl or cetyl citrate; arachidyl propionate; polyvinyl laurate; cholesterol esters, such as triglycerides of plant origin, such as hydrogenated plant oils, viscous polyesters such as poly(12-hydroxystearic acid), and mixtures thereof. Triglycerides of plant origin which may be used are hydrogenated castor oil derivatives, such as “Thixinr” from Rhéox.

Mention may also be made of pasty silicone fatty substances such as polydimethylsiloxanes (PDMSs) containing pendent chains of the alkyl or alkoxy type containing from 8 to 24 carbon atoms, and having a. melting point of 20-55° C., such as stearyldimethicones, in particular those sold by Dow Corning under the trade names DC2503 and DC25514, and mixtures thereof.

The pasty fatty substance may be present in the composition according to the invention in a proportion of from 0% to 60% (in particular 0.01% to 60%) by weight, relative to the total weight of the composition, preferably in a proportion of from 0.5% to 45% by weight, and better still ranging from 2% to 30% by weight, in the composition.

The composition according to the invention may also comprise an aqueous medium, constituting an aqueous phase, which may be the continuous phase of the composition.

The aqueous phase may consist essentially of water; it may also comprise a mixture of water and of water-miscible solvent (miscibility in water of greater than 50% by weight at 25° C.), for instance lower monoalcohols containing from 1 to 5 carbon atoms such as ethanol or isopropanol, glycols containing from 2 to 8 carbon atoms, such as propylene glycol, ethylene glycol, 1,3-butylene glycol or dipropylene glycol, C₃-C₄ ketones and C₂-C₄ aldehydes.

The aqueous phase (water and optionally the water-miscible organic solvent) may be present in a content ranging from 1% to 99% by weight, relative to the total weight of the composition, preferably from 3% to 90% by weight and better still from 5% to 80% by weight.

The composition according to the invention can contain emulsifying surfactants, present in particular in a proportion ranging from 1% to 30% by weight relative to the total weight of the composition, and better still from 5% to 15%. These surfactants may be chosen from anionic and nonionic surfactants. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of the said reference, for the anionic and nonionic surfactants.

The surfactants preferably used in the composition according to the invention are chosen from:

nonionic surfactants: fatty acids, fatty alcohols, polyethoxylated or polyglycerolated fatty alcohols such as polyethoxylated stearyl or cetylstearyl alcohol, fatty acid esters of sucrose, alkylglucose esters, in particular polyoxyethylenated fatty esters of C₁-C₆ alkyl glucose, and mixtures thereof;

anionic surfactants: C₁₆-C₃₀ fatty acids neutralized with amines, aqueous ammonia or alkaline salts, and mixtures thereof.

Surfactants which make it possible to obtain an oil-in-water or wax-in-water emulsion are preferably used.

The composition according to the invention may also comprise a dyestuff, for instance pulverulent dyestuffs, liposoluble dyes and water-soluble dyes. This dyestuff may be present in a content ranging from 0.01% to 30% by weight, relative to the total weight of the composition.

The pulverulent dyestuffs may be chosen from pigments and nacres.

The pigments may be white or coloured, mineral and/or organic, and coated or uncoated. Among the mineral pigments which may be mentioned are titanium dioxide, optionally surface-treated, zirconium oxide, zinc oxide or cerium oxide, as well as iron oxide, chromium oxide, manganese violet, ultramarine blue, chromium hydrate and ferric blue. Among the organic pigments which may be mentioned are carbon black, pigments of D & C type, and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with, in particular, ferric blue or chromium oxide, titanium mica with an organic pigment of the abovementioned type, and nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes are, for example, beetroot juice and methylene blue.

The composition of the invention may also comprise any additive usually used in cosmetics, such as antioxidants, fillers, preserving agents, fragrances, neutralizers, thickeners, cosmetic or dermatological active agents such as, for example, emollients, moisturizers, vitamins and sunscreens, and mixtures thereof. These additives may be present in the composition in a content ranging from 0% to 20% (in particular from 0.01% to 20%) relative to the total weight of the composition and better still from 0.01% to 10% (if present).

Needless to say, a person skilled in the art will take care to select the optional additional additives and/or the amount thereof such that the advantageous properties of the composition according to the invention are not, or are not substantially, adversely affected by the addition envisaged.

The composition according to the invention may be manufactured by the known processes generally used in cosmetics or dermatology.

The invention is illustrated in greater detail in the examples which follow.

### EXAMPLE 1

A mascara having the composition below was prepared:

| | | |
|---|---|---|
| Polyamide resin with ester end groups, | 0 0.5 | g |
| sold under the name “Uniclear ® 100” by | | |
| the company Arizona Chemical | | |
| Carnauba wax | 2.9 | g |
| Beeswax | 3.6 | g |
| Paraffin wax | 11.4 | g |
| 2-Amino-2-methyl-1,3-propanediol | 0.5 | g |
| Triethanolamine | 2.4 | g |
| Stearic acid | 5.8 | g |
| Water-soluble nonionic polymers | 4.3 | g |
| Sodium polymethacrylate (Darvan 7 from | 0.25 | g AM |
| the company Vanderbilt) | | |
| Hydroxyethylcellulose crosslinked with | 0.1 | g |
| epichlorohydrin and quaternized with | | |
| trimethylamine (JR 400 from the company | | |
| Union Carbide) | | |
| Pigments | 5.4 | g |
| Preserving agents qs | | |
| Water qs | 100 | g |

This mascara applies easily, and adheres well to the eyelashes during and after application; the eyelashes are made up quickly. It gives instantaneous loading of the eyelashes.

### EXAMPLE 2

A mascara having the composition below was prepared:

| | | |
|---|---|---|
| Polyamide resin with ester end groups, | 0.5 | g |
| sold under the name “Uniclear ® 100” by | | |
| the company Arizona Chemical | | |
| Carnauba wax | 4.7 | g |
| Beeswax | 4.9 | g |
| Paraffin wax | 2.3 | g |
| Hydroxyethylcellulose crosslinked with | 0.1 | g |
| epichlorohydrin and quaternized with | | |
| trimethylamine (JR 400 from the company | | |
| Union Carbide) | | |
| Sodium polymethacrylate (Darvan 7 from | 0.25 | g AM |
| the company Vanderbilt) | | |
| Sodium deoxyribonucleate | 0.2 | g |
| Water | 8.4 | g |
| Ethyl alcohol | 2 | g |
| Bentonite | 5.3 | g |
| Propylene carbonate | 1.7 | g |
| Vinylpyrrolidone/1-eicosene copolymer | 2 | g |
| Vinyl acetate/allyl stearate copolymer | 2.2 | g |
| (65/35) (Mexomere PQ from Chimex) | | |
| Polyvinyl laurate (Mexomere PP from | 0.7 | g |
| Chimex) | | |
| Rice starch | 1.5 | g |
| Pigments | 4.2 | g |
| Preserving agents qs | | |
| Isododecane qs | 100 | g |

This waterproof mascara adheres well to the eyelashes during and after application. It gives the eyelashes instantaneous loading and allows them to be made up quickly.

1. Composition comprising, in a physiologically acceptable medium containing a fatty phase:
(i) a first polymer with a weight-average molecular mass of less than 100 000, comprising a) a polymer skeleton with hydrocarbon-based repeating units containing at least one hetero atom, and optionally b) optionally functionalized pendent and/or terminal fatty chains containing from 6 to 120 carbon atoms, which are linked to these hydrocarbon-based units,
(ii) an anionic film-forming polymer,
(iii) a cationic film-forming polymer,
the said anionic and cationic film-forming polymers being different from the said first polymer.

2. Composition according to claim 1, characterized in that the average molar mass of the first polymer is less than 50 000.

3. Composition according to claim 1 or 2, characterized in that the units containing a hetero atom of the first polymer are amide groups.

4. Composition according to any one of the preceding claims, characterized in that the fatty chains of the auxiliary polymer represent from 40% to 98% of the total number of units containing a hetero atom and of fatty chains.

5. Composition according to any one of the preceding claims, characterized in that the fatty chains of the first polymer represent from 50% to 95% of the total number of units containing a hetero atom and of fatty chains.

6. Composition according to any one of the preceding claims, characterized in that the pendent fatty chains of the first polymer are linked directly to at least one of the said hetero atoms.

7. Composition containing, in a cosmetically acceptable medium:
(i) a first polyamide polymer with a weight-average molecular mass of less than 100 000, comprising a) a polymer skeleton with amide repeating units and b) optionally at least one optionally functionalized pendent fatty chain and/or at least one optionally functionalized terminal chain, containing from 6 to 120 carbon atoms, which are linked to these amide units,
(ii) an anionic film-forming polymer,
(iii) a cationic film-forming polymer,
the said anionic and cationic film-forming polymers being different from the said first polymer.

8. Composition according to claim 6, characterized in that the fatty chains of the first polymer represent from 40% to 98% of the total number of amide units and of fatty chains.

9. Composition according to any one of claims 6 to 8, characterized in that the fatty chains of the first polymer represent from 50% to 95% of the total number of amide units and of fatty chains.

10. Composition according to any one of claims 6 to 9, characterized in that the pendent fatty chains of the first polymer are linked directly to at least one of the nitrogen atoms of the amide units.

11. Composition according to any one of the preceding claims, characterized in that the weight-average molecular mass of the first polymer ranges from 2000 to 20 000 and better still from 2000 to 10 000.

12. Composition according to any one of the preceding claims, characterized in that the terminal fatty chains of the first polymer are linked to the skeleton via ester groups.

13. Composition according to any one of the preceding claims, characterized in that the fatty chains of the auxiliary polymer contain from 12 to 68 carbon atoms.

14. Composition according to any one of the preceding claims, characterized in that the first polymer is chosen from the polymers of formula (I′) below, and mixtures thereof: 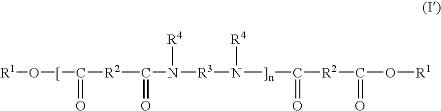
in which n denotes a number of amide units such that the number of ester groups represents from 10% to 50% of the total number of ester and amide groups; R¹ is, independently in each case, an alkyl or alkenyl group containing at least 4 carbon atoms; R² represents, independently in each case, a C₄ to C₄₂ hydrocarbon-based group, on condition that at least 50% of the groups R² represent a C₃₀ to C₄₂ hydrocarbon-based group; R³ represents, independently in each case, an organic group containing at least 2 carbon atoms, hydrogen atoms and optionally one or more oxygen or nitrogen atoms; and R⁴ represents, independently in each case, a hydrogen atom, a C₁ to C₁₀ alkyl group or a direct bond to R³ or to another R⁴, such that the nitrogen atom to which R³ and R⁴ are both attached forms part of a heterocyclic structure defined by R⁴—N—R³, with at least 50% of the groups R⁴ representing a hydrogen atom.

15. Composition according to claim 14, characterized in that R¹ is a C₁₂ to C₂₂ alkyl group.

16. Composition according to claim 14 or 15, characterized in that the radicals R² are groups containing from 30 to 42 carbon atoms.

17. Composition according to any one of the preceding claims, characterized in that the first polymer is present in a content ranging from 0.01% to 10% by weight, preferably ranging from 0.05% to 5% by weight and better still ranging from 0.1% to 3% by weight, relative to the total weight of the composition.

18. Composition according to any one of the preceding claims, characterized in that the anionic film-forming polymer is chosen from:
polymers comprising carboxylic units derived from unsaturated monocarboxylic or dicarboxylic acid monomers of formula (I): 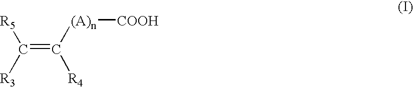
in which n is an integer from 0 to 10, A denotes a methylene group, optionally connected to the carbon atom of the unsaturated group or to the neighbouring methylene group when n is greater than 1 via a hetero atom such as oxygen or sulphur, R₅ denotes a hydrogen atom or a phenyl or benzyl group, R₃ denotes a hydrogen atom or a lower alkyl or carboxyl group, and R₄ denotes a hydrogen atom, a lower alkyl group or a —CH₂—COOH, phenyl or benzyl group,
polymers comprising units derived from sulphonic acid, such as vinylsulphonic, styrenesulphonic and acrylamidoalkylsulphonic units, and sulphonic polyesters, and
mixtures thereof.

19. Composition according to any one of the preceding claims, characterized in that the anionic film-forming polymer is chosen from:
A) homo- or copolymers of acrylic or methacrylic acid or salts thereof, the sodium salts of copolymers of acrylic acid and of acrylamide, and the sodium salts of polyhydroxycarboxylic acids;
B) copolymers of acrylic or methacrylic acids with a monoethylenic monomer such as ethylene, styrene, vinyl esters and acrylic or methacrylic acid esters, optionally grafted onto a polyalkylene glycol such as polyethylene glycol; copolymers of this type comprising in their chain an optionally N-alkylated and/or hydroxyalkylated acrylamide unit, copolymers of acrylic acid and of C₁-C₄ alkyl methacrylate and terpolymers of vinylpyrrolidone, of acrylic acid and of C₁-C₂₀ alkyl methacrylate;
C) copolymers derived from crotonic acid, such as those whose chain comprises vinyl acetate or propionate units and optionally other monomers such as allylic or methallylic esters, vinyl ether or vinyl ester of a saturated, linear or branched carboxylic acid containing a long hydrocarbon-based chain such as those comprising at least 5 carbon atoms, it being possible for these polymers to be optionally grafted;
D) polymers derived from maleic, fumaric or itaconic acids or anhydrides with vinyl esters, vinyl ethers, vinyl halides, phenylvinyl derivatives, acrylic acid and esters thereof; copolymers of maleic, citraconic or itaconic anhydrides and of an allylic or methallylic ester optionally comprising an acrylamide, methacrylamide, α-olefin, acrylic or methacrylic ester, acrylic or methacrylic acid or vinylpyrrolidone group in their chain, the anhydride functions are monoesterified or monoamidated;
E) polyacrylamides comprising carboxylate groups,
F) deoxyribonucleic acid;
G) copolymers-of at least one dicarboxylic acid, of at least one diol and of at least one difunctional aromatic monomer bearing a group —SO₃M with M representing a hydrogen atom, an ammonium ion NH₄⁺ or a metal ion;
and mixtures thereof.

20. Composition according to any one of the preceding claims, characterized in that the anionic film-forming polymer is chosen from:
acrylic or methacrylic acid homopolymers;
acrylic acid copolymers such as the acrylic acid/ethyl acrylate/N-tert-butylacrylamide terpolymer;
copolymers derived from crotonic acid, such as vinyl acetate/vinyl tert-butylbenzoate/crotonic acid terpolymers and crotonic acid/vinyl acetate/vinyl neododecanoate terpolymers;
polymers derived from maleic, fumaric or itaconic acids or anhydrides with vinyl esters, vinyl ethers, vinyl halides, phenylvinyl derivatives or acrylic acid and esters thereof, such as methyl vinyl ether/monoesterified maleic anhydride copolymers;
copolymers of methacrylic acid and of methyl methacrylate;
copolymers of methacrylic acid and of ethyl acrylate;
terpolymers of vinylpyrrolidone/acrylic acid/lauryl methacrylate;
vinyl acetate/crotonic acid copolymers;
vinyl acetate/crotonic acid/polyethylene glycol terpolymers;
sulphopolyesters obtained by condensation of diethylene glycol, cyclohexanedimethanol, isophthalic acid and sulphoisophthalic acid,
and mixtures thereof.

21. Composition according to any one of the preceding claims, characterized in that the anionic film-forming polymer is chosen from anionic polymers of grafted silicone type comprising a polysiloxane portion and a portion consisting of a non-silicone organic chain, one of the two portions constituting the main chain of the polymer, the other being grafted onto the said main chain.

22. Composition according to claim 21, characterized in that the grafted silicone polymer is chosen from silicone polymers whose structure comprises the unit of formula (III) below: 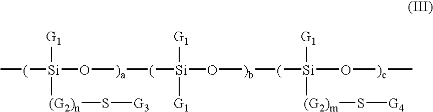
in which the radicals G₁, which may be identical or different, represent hydrogen or a C₁-C₁₀ alkyl radical or alternatively a phenyl radical; the radicals G₂, which may be identical or different, represent a C₁-C₁₀ alkylene group; G₃ represents a polymer residue resulting from the (homo)polymerization of at least one ethylenically unsaturated anionic monomer; G₄ represents a polymer residue resulting from the (homo)polymerization of at least one ethylenically unsaturated hydrophobic monomer; m and n are equal to 0 or 1; a is an integer ranging from 0 to 50; b is an integer which can be between 10 and 350, c is an integer ranging from 0 to 50; with the proviso that one of the parameters a and c is other than 0.

23. Composition according to claim 22, characterized in that the unit of formula (III) has at least one of the following characteristics:
the radicals G₁ denote a C₁-C₁₀ alkyl radical;
n is non-zero and the radicals G₂ represent a divalent C₁-C₃ radical;
G₃ represents a polymer radical resulting from the (homo)polymerization of at least one monomer such as an ethylenically unsaturated carboxylic acid;
G₄ represents a polymer radical resulting from the (homo)polymerization of at least one monomer such as a C₁-C₁₀ alkyl (meth)acrylate.

24. Composition according to claim 22 or 23, characterized in that the unit of formula (III) simultaneously has the following characteristics:
the radicals G₁ denote a methyl radical;
n is non-zero and the radicals G₂ represent a propylene radical;
G₃ represents a polymer radical resulting from the (homo)polymerization of at least acrylic acid and/or methacrylic acid;
G4 represents a polymer radical resulting from the (homo)polymerization of at least isobutyl or methyl (meth)acrylate.

25. Composition according to any one of the preceding claims, characterized in that the cationic film-forming polymer is chosen from quaternary cellulose ether derivatives, copolymers of cellulose with a water-soluble quaternary ammonium monomer, cyclopolymers, cationic polysaccharides, cationic silicone polymers, quaternized or non-quaternized vinylpyrrolidone-dialkylaminoalkyl acrylate or methacrylate copolymers, quaternary polymers of vinylpyrrolidone and of vinylimidazole, and polyaminoamides, and mixtures thereof.

26. Composition according to any one of the preceding claims, characterized in that the anionic film-forming polymer is a poly(sodium methacrylate).

27. Composition according to any one of the preceding claims, characterized in that the cationic film-forming polymer is a hydroxy (C₁-C₄)alkylcellulose comprising quaternary ammonium groups.

28. Composition according to any one of the preceding claims, characterized in that the cationic film-forming polymer is present in a content ranging from 0.01% to 20% by weight, preferably from 0.01% to 15% by weight and even more preferentially from 0.05% to 5% by weight, relative to the total weight of the composition.

29. Composition according to any one of the preceding claims, characterized in that the anionic film-forming polymer is present in a content ranging from 0.01% to 20% by weight, preferably from 0.05% to 15% by weight and even more preferentially from 0.1% to 7% by weight, relative to the total weight of the composition.

30. Composition according to any one of the preceding claims, characterized in that it also comprises a wax.

31. Composition according to claim 30, characterized in that the wax is chosen from the group formed by beeswax, lanolin wax, Chinese insect waxes, rice wax, carnauba wax, candelilla wax, ouricury wax, cork fibre wax, sugar cane wax, Japan wax, sumach wax, montan wax, microcrystalline waxes, paraffin waxes, ozokerites, ceresin wax, lignite wax, polyethylene waxes and the waxes obtained by Fisher-Tropsch synthesis, fatty acid esters of glycerides that are solid at 40° C., the waxes obtained by catalytic hydrogenation of animal or plant oils containing linear or branched C₈-C₃₂ fatty chains, silicone waxes and fluoro waxes, and mixtures thereof.

32. Composition according to claim 30 or 31, characterized in that the wax is present in a content ranging from 0.1% to 50% by weight, preferably from 0.5% to 40% by weight and better still from 1% to 30% by weight, relative to the total weight of the composition.

33. Composition according to any one of the preceding claims, characterized in that the fatty phase comprises at least one oil chosen from the group formed by hydrocarbon-based oils, fluoro oils and/or silicone oils of mineral, animal, plant or synthetic origin, alone or as a mixture.

34. Composition according to any one of the preceding claims, characterized in that the fatty phase comprises at least one volatile oil.

35. Composition according to any one of the preceding claims, characterized in that the fatty phase comprises a volatile oil chosen from hydrocarbon-based volatile oils containing from 8 to 16 carbon atoms.

36. Composition according to claim 34 or 35, characterized in that the volatile oil is present in a content ranging from 0.1% to 98% by weight and preferably from 1% to 65% by weight, relative to the total weight of the composition.

37. Composition according to any one of the preceding claims, characterized in that the composition comprises an aqueous phase containing water or a mixture of water and of water-miscible organic solvent.

38. Composition according to any one of the preceding claims, characterized in that the composition contains at least one dyestuff.

39. Composition according to claim 38, characterized in that the dyestuff is chosen from pigments, nacres, water-soluble dyes and liposoluble dyes, and mixtures thereof.

40. Composition according to claim 38 or 39, characterized in that the dyestuff is present in a proportion of from 0.01% to 30% of the total weight of the composition.

41. Composition according to any one of the preceding claims, characterized in that the composition contains at least one additive chosen from surfactants, thickeners, antioxidants, fillers, preserving agents, fragrances, neutralizers and cosmetic or dermatological active agents, and mixtures thereof.

42. Composition according to any one of the preceding claims, characterized in that the composition is in the form of a mascara, a product for the eyebrows or a product for the hair.

43. Mascara comprising a composition according to any one of claims 1 to 41.

44. Non-therapeutic makeup or care process for keratin materials, especially keratin fibres, comprising the application to the keratin materials of a composition according to any one of the preceding claims.

45. Use of a composition according to any one of claims 1 to 42, to obtain a deposit that adheres to keratin materials and/or to obtain a fast makeup result on keratin materials.

46. Use of a mascara according to claim 43, to thicken the eyelashes.

47. Use of the combination of
(i) a first polymer with a weight-average molecular mass of less than 100 000, comprising a) a polymer skeleton with hydrocarbon-based repeating units containing at least one hetero atom, and optionally b) optionally functionalized pendent and/or terminal fatty chains containing from 6 to 120 carbon atoms, which are linked to these hydrocarbon-based units,
(ii) an anionic film-forming polymer,
(iii) a cationic film-forming polymer,
the said anionic and cationic film-forming polymers being different from the said first polymer, in a makeup composition comprising a physiologically acceptable medium containing a fatty phase, to obtain a deposit that adheres to the keratin materials and/or a fast makeup result on keratin materials and/or to thicken the eyelashes.

48. Use according to claim 47, characterized in that the average molar mass of the first polymer is less than 50 000.

49. Use according to claim 47 or 48, characterized in that the units containing a hetero atom of the first polymer are amide groups.

50. Use according to any one of claims 47 to 49, characterized in that the fatty chains of the auxiliary polymer represent from 40% to 98% of the total number of units containing a hetero atom and of fatty chains.

51. Use according to any one of claims 47 to 50, characterized in that the fatty chains of the first polymer represent from 50% to 95% of the total number of units containing a hetero atom and of fatty chains.

52. Use according to any one of claims 47 to 51, characterized in that the pendent fatty chains of the first polymer are linked directly to at least one of the said hetero atoms.

53. Use of the combination of:
(i) a first polyamide polymer with a weight-average molecular mass of less than 100 000, comprising a) a polymer skeleton with amide repeating units and b) optionally at least one optionally functionalized pendent fatty chain and/or at least one optionally functionalized terminal chain, containing from 6 to 120 carbon atoms, which are linked to these amide units,
(ii) an anionic film-forming polymer,
(iii) a cationic film-forming polymer,
the said anionic and cationic film-forming polymers being different from the said first polymer, to obtain a deposit that adheres to the keratin materials and/or a fast makeup result on keratin materials and/or to thicken the eyelashes.

54. Use according to claim 53, characterized in that the fatty chains of the first polymer represent from 40% to 98% of the total number of amide units and of fatty chains.

55. Use according to either of claims 53 and 54, characterized in that the fatty chains of the first polymer represent from 50% to 95% of the total number of amide units and of fatty chains.

56. Use according to any one of claims 53 to 55, characterized in that the pendent fatty chains of the first polymer are linked directly to at least one of the nitrogen atoms of the amide units.

57. Use according to any one of claims 47 to 56, characterized in that the weight-average molecular mass of the first polymer ranges from 2000 to 20 000 and better still from 2000 to 10 000.

58. Use according to any one of claims 47 to 57, characterized in that the terminal fatty chains of the first polymer are linked to the skeleton via ester groups.

59. Use according to any one of claims 47 to 58, characterized in that the fatty chains of the auxiliary polymer contain from 12 to 68 carbon atoms.

60. Use according to any one of claims 47 to 59, characterized in that the first polymer is chosen from the polymers of formula (I′) below, and mixtures thereof: 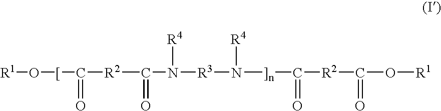
in which n denotes a number of amide units such that the number of ester groups represents from 10% to 50% of the total number of ester and amide groups; R¹ is, independently in each case, an alkyl or alkenyl group containing at least 4 carbon atoms; R² represents, independently in each case, a C₄ to C₄₂ hydrocarbon-based group, on condition that at least 50% of the groups R² represent a C₃₀ to C₄₂ hydrocarbon-based group; R³ represents, independently in each case, an organic group containing at least 2 carbon atoms, hydrogen atoms and optionally one or more oxygen or nitrogen atoms; and R⁴ represents, independently in each case, a hydrogen atom, a C₁ to C₁₀ alkyl group or a direct bond to R³ or to another R⁴, such that the nitrogen atom to which R³ and R⁴ are both attached forms part of a heterocyclic structure defined by R⁴—N—R³, with at least 50% of the groups R⁴ representing a hydrogen atom.

61. Use according to claim 60, characterized in that R¹ is a C₁₂ to C₂₂ alkyl group.

62. Use according to claim 60 or 61, characterized in that the radicals R² are groups containing from 30 to 42 carbon atoms.

63. Use according to any one of claims 47 to 62, characterized in that the first polymer is present in the composition in a content ranging from 0.01% to 10% by weight, preferably ranging from 0.05% to 5% by weight and better still ranging from 0.1% to 3% by weight, relative to the total weight of the composition.

64. Use according to any one of claims 47 to 63, characterized in that the anionic film-forming polymer is chosen from:
polymers comprising carboxylic units derived from unsaturated monocarboxylic or dicarboxylic acid monomers of formula (I): 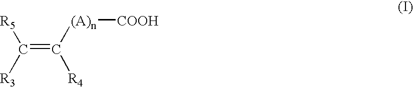
in which n is an integer from 0 to 10, A denotes a methylene group, optionally connected to the carbon atom of the unsaturated group or to the neighbouring methylene group when n is greater than 1 via a hetero atom such as oxygen or sulphur, R₅ denotes a hydrogen atom or a phenyl or benzyl group, R₃ denotes a hydrogen atom or a lower alkyl or carboxyl group, and R₄ denotes a hydrogen atom, a lower alkyl group or a —CH₂—COOH, phenyl or benzyl group,
polymers comprising units derived from sulphonic acid, such as vinylsulphonic, styrenesulphonic and acrylamidoalkylsulphonic units, and sulphonic polyesters, and
mixtures thereof.

65. Use according to any one of claims 47 to 64, characterized in that the anionic film-forming polymer is chosen from:
A) homo- or copolymers of acrylic or methacrylic acid or salts thereof, the sodium salts of copolymers of acrylic acid and of acrylamide, and the sodium salts of polyhydroxycarboxylic acids;
B) copolymers of acrylic or methacrylic acids with a monoethylenic monomer such as ethylene, styrene, vinyl esters and acrylic or methacrylic acid esters, optionally grafted onto a polyalkylene glycol such as polyethylene glycol; copolymers of this type comprising in their chain an optionally N-alkylated and/or hydroxyalkylated acrylamide unit, copolymers of acrylic acid and of C₁-C₄ alkyl methacrylate and terpolymers of vinylpyrrolidone, of acrylic acid and of C₁-C₂₀ alkyl methacrylate;
C) copolymers derived from crotonic acid, such as those whose chain comprises vinyl acetate or propionate units and optionally other monomers such as allylic or methallylic esters, vinyl ether or vinyl ester of a saturated, linear or branched carboxylic acid containing a long hydrocarbon-based chain such as those comprising at least 5 carbon atoms, it being possible for these polymers to be optionally grafted;
D) polymers derived from maleic, fumaric or itaconic acids or anhydrides with vinyl esters, vinyl ethers, vinyl halides, phenylvinyl derivatives, acrylic acid and esters thereof; copolymers of maleic, citraconic or itaconic anhydrides and of an allylic or methallylic ester optionally comprising an acrylamide, methacrylamide, α-olefin, acrylic or methacrylic ester, acrylic or methacrylic acid or vinylpyrrolidone group in their chain, the anhydride functions are monoesterified or monoamidated;
E) polyacrylamides comprising carboxylate groups,
F) deoxyribonucleic acid;
G) copolymers of at least one dicarboxylic acid, of at least one diol and of at least one difunctional aromatic monomer bearing a group —SO₃M with M representing a hydrogen atom, an ammonium ion NH₄⁺ or a metal ion;
and mixtures thereof.

66. Use according to any one of claims 47 to 65, characterized in that the anionic film-forming polymer is chosen from:
acrylic or methacrylic acid homopolymers;
acrylic acid copolymers such as the acrylic acid/ethyl acrylate/N-tert-butylacrylamide terpolymer;
copolymers derived from crotonic acid, such as vinyl acetate/vinyl tert-butylbenzoate/crotonic acid terpolymers and crotonic acid/vinyl acetate/vinyl neododecanoate terpolymers;
polymers derived from maleic, fumaric or itaconic acids or anhydrides with vinyl esters, vinyl ethers, vinyl halides, phenylvinyl derivatives or acrylic acid and esters thereof, such as methyl vinyl ether/monoesterified maleic anhydride copolymers;
copolymers of methacrylic acid and of methyl methacrylate;
copolymers of methacrylic acid and of ethyl acrylate;
terpolymers of vinylpyrrolidone/acrylic acid/lauryl methacrylate;
vinyl acetate/crotonic acid copolymers;
vinyl acetate/crotonic acid/polyethylene glycol terpolymers;
sulphopolyesters obtained by condensation of diethylene glycol, cyclohexanedimethanol, isophthalic acid and sulphoisophthalic acid,
and mixtures thereof.

67. Use according to any one of claims 47 to 66, characterized in that the anionic film-forming polymer is chosen from anionic polymers of grafted silicone type comprising a polysiloxane portion and a portion consisting of a non-silicone organic chain, one of the two portions constituting the main chain of the polymer, the other being grafted onto the said main chain.

68. Use according to claim 67, characterized in that the grafted silicone polymer is chosen from silicone polymers whose structure comprises the unit of formula (III) below: 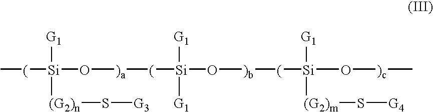
in which the radicals G₁, which may be identical or different, represent hydrogen or a C₁-C₁₀ alkyl radical or alternatively a phenyl radical; the radicals G₂, which may be identical or different, represent a C₁-C₁₀ alkylene group; G₃ represents a polymer residue resulting from the (homo)polymerization of at least one ethylenically unsaturated anionic monomer; G₄ represents a polymer residue resulting from the (homo)polymerization of at least one ethylenically unsaturated hydrophobic monomer; m and n are equal to 0 or 1; a is an integer ranging from 0 to 50; b is an integer which can be between 10 and 350, c is an integer ranging from 0 to 50; with the proviso that one of the parameters a and c is other than 0.

69. Use according to claim 68, characterized in that the unit of formula (III) has at least one of the following characteristics:
the radicals G₁ denote a C₁-C₁₀ alkyl radical;
n is non-zero and the radicals G₂ represent a divalent C₁-C₃ radical;
G₃ represents a polymer radical resulting from the (homo)polymerization of at least one monomer such as an ethylenically unsaturated carboxylic acid;
G₄ represents a polymer radical resulting from the (homo)polymerization of at least one monomer such as a C₁-C₁₀ alkyl (meth)acrylate.

70. Use according to claim 68 or 69, characterized in that the unit of formula (III) simultaneously has the following characteristics:
the radicals G₁ denote a methyl radical;
n is non-zero and the radicals G₂ represent a propylene radical;
G₃ represents a polymer radical resulting from the (homo)polymerization of at least acrylic acid and/or methacrylic acid;
G₄ represents a polymer radical resulting from the (homo)polymerization of at least isobutyl or methyl (meth)acrylate.

71. Use according to any one of claims 47 to 70, characterized in that the cationic film-forming polymer is chosen from quaternary cellulose ether derivatives, copolymers of cellulose with a water-soluble quaternary ammonium monomer, cyclopolymers, cationic polysaccharides, cationic silicone polymers, quaternized or non-quaternized vinylpyrrolidone-dialkylaminoalkyl acrylate or methacrylate copolymers, quaternary polymers of vinylpyrrolidone and of vinylimidazole, and polyaminoamides, and mixtures thereof.

72. Use according to any one of claims 47 to 71, characterized in that the anionic film-forming polymer is a poly(sodium methacrylate).

73. Use according to any one of claims 47 to 72, characterized in that the cationic film-forming polymer is a hydroxy(C₁-C₄)alkylcellulose comprising quaternary ammonium groups.

74. Use according to any one of claims 47 to 73, characterized in that the cationic film-forming polymer is present in the composition in a content ranging from 0.01% to 20% by weight, preferably from 0.01% to 15% by weight and even more preferentially from 0.05% to 5% by weight, relative to the total weight of the composition.

75. Use according to any one of claims 47 to 74, characterized in that the anionic film-forming polymer is present in the composition in a content ranging from 0.01% to 20% by weight, preferably from 0.05% to 15% by weight and even more preferentially from 0.1% to 7% by weight, relative to the total weight of the composition.

76. Use according to any one of claims 47 to 75, characterized in that the composition comprises a wax.

77. Use according to claim 76, characterized in that the wax is chosen from the group formed by beeswax, lanolin wax, Chinese insect waxes, rice wax, carnauba wax, candelilla wax, ouricury wax, cork fibre wax, sugar cane wax, Japan wax, sumach wax, montan wax, microcrystalline waxes, paraffin waxes, ozokerites, ceresin wax, lignite wax, polyethylene waxes and the waxes obtained by Fisher-Tropsch synthesis, fatty acid esters of glycerides that are solid at 40° C., the waxes obtained by catalytic hydrogenation of animal or plant oils containing linear or branched C₈-C₃₂ fatty chains, silicone waxes and fluoro waxes, and mixtures thereof.

78. Use according to claim 76 or 77, characterized in that the wax is present in a content ranging from 0.1% to 50% by weight, preferably from 0.5% to 40% by weight and better still from 1% to 30% by weight, relative to the total weight of the composition.

79. Use according to any one of claims 47 to 78, characterized in that the fatty phase comprises at least one oil chosen from the group formed by hydrocarbon-based oils, fluoro oils and/or silicone oils of mineral, animal, plant or synthetic origin, alone or as a mixture.

80. Use according to any one of claims 47 to 79, characterized in that the fatty phase comprises at least one volatile oil.

81. Use according to any one of claims 45 to 80, characterized in that the fatty phase comprises a volatile oil chosen from hydrocarbon-based volatile oils containing from 8 to 16 carbon atoms.

82. Use according to claim 80 or 81, characterized in that the volatile oil is present in a content ranging from 0.1% to 98% by weight and preferably from 1% to 65% by weight, relative to the total weight of the composition.

83. Use according to any one of claims 47 to 82, characterized in that the composition comprises an aqueous phase containing water or a mixture of water and of water-miscible organic solvent.

84. Use according to any one of claims 47 to 83, characterized in that the composition contains at least one additive chosen from dyestuffs, surfactants, thickeners, antioxidants, fillers, preserving agents, fragrances, neutralizers and cosmetic or dermatological active agents, and mixtures thereof.

85. Use according to any one of claims 47 to 84, characterized in that the composition is in the form of a mascara, a product for the eyebrows or a product for the hair.

86. Cosmetic process for rapidly making up keratin materials, which consists in introducing, into a cosmetic makeup composition comprising a fatty phase:
(i) a first polymer with a weight-average molecular mass of less than 100 000, comprising a) a polymer skeleton with hydrocarbon-based repeating units containing at least one hetero atom, and optionally b) optionally functionalized pendent and/or terminal fatty chains containing from 6 to 120 carbon atoms, which are linked to these hydrocarbon-based units,
(ii) an anionic film-forming polymer,
(iii) a cationic film-forming polymer,
the said anionic and cationic film-forming polymers being different from the said first polymer.

87. Cosmetic process for increasing the adhesion and/or the rapid loading of a cosmetic makeup composition, which consists in introducing into the said composition containing a fatty phase:
(i) a first polymer with a weight-average molecular mass of less than 100 000, comprising a) a polymer skeleton with hydrocarbon-based repeating units containing at least one hetero atom, and optionally b) optionally functionalized pendent and/or terminal fatty chains containing from 6 to 120 carbon atoms, which are linked to these hydrocarbon-based units,
(ii) an anionic film-forming polymer,
(iii) a cationic film-forming polymer, the said anionic and cationic film-forming polymers being different from the said first polymer.

88. Process according to claim 86 or 87, characterized in that the average molar mass of the first polymer is less than 50 000.

89. Process according to any one of claims 86 to 88, characterized in that the units containing a hetero atom of the first polymer are amide groups.

90. Process according to any one of claims 86 to 89, characterized in that the fatty chains represent from, 40% to 98% and better still from 50% to 95% of the total number of units containing a hetero atom and of fatty chains.

91. Process according to any one of claims 86 to 90, characterized in that the fatty chains represent from 50% to 95% of the total number of units containing a hetero atom and of fatty chains.

92. Process according to any one of claims 86 to 91, characterized in that the pendent fatty chains are linked directly to at least one of the said hetero atoms.

93. Cosmetic process for rapidly making up keratin materials, which consists in introducing, into a cosmetic makeup composition comprising a fatty phase:
(i) a first polyamide polymer with a weight-average molecular mass of less than 100 000, comprising a) a polymer skeleton with amide repeating units and b) optionally at least one optionally functionalized pendent fatty chain and/or at least one optionally functionalized terminal chain, containing from 6 to 120 carbon atoms, which are linked to these amide units,
(ii) an anionic film-forming polymer,
(iii) a cationic film-forming polymer,
the said anionic and cationic film-forming polymers being different from the said first polymer.

94. Cosmetic process for increasing the adhesion and/or the rapid loading of a cosmetic makeup composition, which consists in introducing into the said composition containing a fatty phase:
(i) a first polyamide polymer with a weight-average molecular mass of less than 100 000, comprising a) a polymer skeleton with amide repeating units and b) optionally at least one optionally functionalized pendent fatty chain and/or at least one optionally functionalized terminal chain, containing from 6 to 120 carbon atoms, which are linked to these amide units,
(ii) an anionic film-forming polymer,
(iii) a cationic film-forming polymer,
the said anionic and cationic film-forming polymers being different from the said first polymer.

95. Process according to claim 93 or 94, characterized in that the fatty chains of the first polymer represent from 40% to 98% of the total number of amide units and of fatty chains.

96. Process according to any one of claims 93 to 95, characterized in that the fatty chains of the first polymer represent from 50% to 95% of the total number of amide units and of fatty chains.

97. Process according to any one of claims 93 to 96, characterized in that the pendent fatty chains are linked directly to at least one of the nitrogen atoms of the amide units.

98. Process according to any one of claims 86 to 97, characterized in that the weight-average molecular mass of the first polymer ranges from 1000 to 100 000, preferably from 1000 to 50 000 and better still from 1000 to 30 000.

99. Process according to one of claims 86 to 98, characterized in that the weight-average molar mass of the first film-forming polymer ranges from 2000 to 20 000 and preferably from 2000 to 10 000.

100. Process according to one of claims 86 to 99, characterized in that the terminal fatty chain(s) is (are) linked to the skeleton via bonding groups.

101. Process according to claim 100, characterized in that the bonding groups are ester groups.

102. Process according to any one of claims 86 to 101, characterized in that the fatty chains contain from 12 to 68 carbon atoms.

103. Process according to any one of claims 86 to 102, characterized in that the first polymer is chosen from the polymers of formula (I′) below, and mixtures thereof: 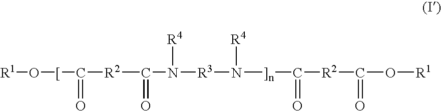
in which n denotes a number of amide units such that the number of ester groups represents from 10% to 50% of the total number of ester and amide groups; R¹ is, independently in each case, an alkyl or alkenyl group containing at least 4 carbon atoms; R² represents, independently in each case, a C₄ to C₄₂ hydrocarbon-based group, on condition that at least 50% of the groups R² represent a C₃₀ to C₄₂ hydrocarbon-based group; R³ represents, independently in each case, an organic group containing at least 2 carbon atoms, hydrogen atoms and optionally one or more oxygen or nitrogen atoms; and R⁴ represents, independently in each case, a hydrogen atom, a C₁ to C₁₀ alkyl group or a direct bond to R³ or another R⁴, such that the nitrogen atom to which R³ and R⁴ are both attached forms part of a heterocyclic structure defined by R⁴—N—R³, with at least 50% of the groups R⁴ representing a hydrogen atom.

104. Process according to claim 103, characterized in that R¹ is a C₁₂ to C₂₂ alkyl group.

105. Process according to claim 103 or 104, characterized in that the radicals R² are groups containing from 30 to 42 carbon atoms.

106. Process according to any one of claims 86 to 105, characterized in that the first polymer is present in a content ranging from 0.01% to 10% by weight, preferably ranging from 0.05% to 5% by weight and better still ranging from 0.1% to 3% by weight, relative to the total weight of the composition.

107. Process according to any one of claims 86 to 106, characterized in that the anionic film-forming polymer is chosen from:
polymers comprising carboxylic units derived from unsaturated monocarboxylic or dicarboxylic acid monomers of formula (I): 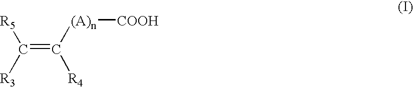
in which n is an integer from 0 to 10, A denotes a methylene group, optionally connected to the carbon atom of the unsaturated group or to the neighbouring methylene group when n is greater than 1 via a hetero atom such as oxygen or sulphur, R₅ denotes a hydrogen atom or a phenyl or benzyl group, R₃ denotes a hydrogen atom or a lower alkyl or carboxyl group, and R₄ denotes a hydrogen atom, a lower alkyl group or a —CH₂—COOH, phenyl or benzyl group,
polymers comprising units derived from sulphonic acid, such as vinylsulphonic, styrenesulphonic and acrylamidoalkylsulphonic units, and sulphonic polyesters, and
mixtures thereof.

108. Process according to any one of claims 86 to 107, characterized in that the anionic film-forming polymer is chosen from:
A) homo- or copolymers of acrylic or methacrylic acid or salts thereof, the sodium salts of copolymers of acrylic acid and of acrylamide, and the sodium salts of polyhydroxycarboxylic acids;
B) copolymers of acrylic or methacrylic acids with a monoethylenic monomer such as ethylene, styrene, vinyl esters and acrylic or methacrylic acid esters, optionally grafted onto a polyalkylene glycol such as polyethylene glycol; copolymers of this type comprising in their chain an optionally N-alkylated and/or hydroxyalkylated acrylamide unit, copolymers of acrylic acid and of C₁-C₄ alkyl methacrylate and terpolymers of vinylpyrrolidone, of acrylic acid and of C₁-C₂₀ alkyl methacrylate;
C) copolymers derived from crotonic acid, such as those whose chain comprises vinyl acetate or propionate units and optionally other monomers such as allylic or methallylic esters, vinyl ether or vinyl ester of a saturated, linear or branched carboxylic acid containing a long hydrocarbon-based chain such as those comprising at least 5 carbon atoms, it being possible for these polymers to be optionally grafted;
D) polymers derived from maleic, fumaric or itaconic acids or anhydrides with vinyl esters, vinyl ethers, vinyl halides, phenylvinyl derivatives, acrylic acid and esters thereof; copolymers of maleic, citraconic or itaconic anhydrides and of an allylic or methallylic ester optionally comprising an acrylamide, methacrylamide, α-olefin, acrylic or methacrylic ester, acrylic or methacrylic acid or vinylpyrrolidone group in their chain, the anhydride functions are monoesterified or monoamidated;
E) polyacrylamides comprising carboxylate groups,
F) deoxyribonucleic acid;
G) copolymers of at least one dicarboxylic acid, of at least one diol and of at least one difunctional aromatic monomer bearing a group —SO₃M with M representing a hydrogen atom, an ammonium ion NH₄⁺ or a metal ion;
and mixtures thereof.

109. Use according to any one of claims 86 to 108, characterized in that the anionic film-forming polymer is chosen from:
acrylic or methacrylic acid homopolymers;
acrylic acid copolymers such as the acrylic acid/ethyl acrylate/N-tert-butylacrylamide terpolymer;
copolymers derived from crotonic acid, such as vinyl acetate/vinyl tert-butylbenzoate/crotonic acid terpolymers and crotonic acid/vinyl acetate/vinyl neododecanoate terpolymers;
polymers derived from maleic, fumaric or itaconic acids or anhydrides with vinyl esters, vinyl ethers, vinyl halides, phenylvinyl derivatives or acrylic acid and esters thereof, such as methyl vinyl ether/monoesterified maleic anhydride copolymers;
copolymers of methacrylic acid and of methyl methacrylate;
copolymers of methacrylic acid and of ethyl acrylate;
terpolymers of vinylpyrrolidone/acrylic acid/lauryl methacrylate;
vinyl acetate/crotonic acid copolymers;
vinyl acetate/crotonic acid/polyethylene glycol terpolymers;
sulphopolyesters obtained by condensation of diethylene glycol, cyclohexanedimethanol, isophthalic acid and sulphoisophthalic acid,
and mixtures thereof.

110. Process according to any one of claims 86 to 109, characterized in that the anionic film-forming polymer is chosen from anionic polymers of grafted silicone type comprising a polysiloxane portion and a portion consisting of a non-silicone organic chain, one of the two portions constituting the main chain of the polymer, the other being grafted onto the said main chain.

111. Process according to claim 110, characterized in that the grafted silicone polymer is chosen from silicone polymers whose structure comprises the unit of formula (III) below: 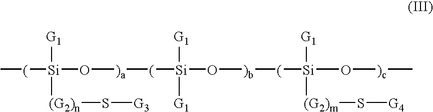
in which the radicals G₁, which may be identical or different, represent hydrogen or a C₁-C₁₀ alkyl radical or alternatively a phenyl radical; the radicals G₂, which may be identical or different, represent a C₁-C₁₀ alkylene group; G₃ represents a polymer residue resulting from the (homo)polymerization of at least one ethylenically unsaturated anionic monomer; G₄ represents a polymer residue resulting from the (homo)polymerization of at least one ethylenically unsaturated hydrophobic monomer; m and n are equal to 0 or 1; a is an integer ranging from 0 to 50; b is an integer which can be between 10 and 350, c is an integer ranging from 0 to 50; with the proviso that one of the parameters a and c is other than 0.

112. Process according to claim 111, characterized in that the unit of formula (III) has at least one of the following characteristics:
the radicals G₁ denote a C₁-C₁₀ alkyl radical;
n is non-zero and the radicals G₂ represent a divalent C₁-C₃ radical;
G₃ represents a polymer radical resulting from the (homo)polymerization of at least one monomer such as an ethylenically unsaturated carboxylic acid;
G₄ represents a polymer radical resulting from the (homo)polymerization of at least one monomer such as a C₁-C₁₀ alkyl (meth)acrylate.

113. Process according to claim 111 or 112, characterized in that the unit of formula (III) simultaneously has the following characteristics:
the radicals G₁ denote a methyl radical;
n is non-zero and the radicals G₂ represent a propylene radical;
G₃ represents a polymer radical resulting from the (homo)polymerization of at least acrylic acid and/or methacrylic acid;
G₄ represents a polymer radical resulting from the (homo)polymerization of at least isobutyl or methyl (meth)acrylate.

114. Process according to any one of claims 86 to 113, characterized in that the cationic film-forming polymer is chosen from quaternary cellulose ether derivatives, copolymers of cellulose with a water-soluble quaternary ammonium monomer, cyclopolymers, cationic polysaccharides, cationic silicone polymers, quaternized or non-quaternized vinylpyrrolidone-dialkylaminoalkyl acrylate or methacrylate copolymers, quaternary polymers of vinylpyrrolidone and of vinylimidazole, and polyaminoamides, and mixtures thereof.

115. Process according to any one of claims 86 to 114, characterized in that the anionic film-forming polymer is a poly(sodium methacrylate).

116. Process according to any one of claims 86 to 115, characterized in that the cationic film-forming polymer is a hydroxy(C₁-C₄)alkylcellulose comprising quaternary ammonium groups.

117. Process according to any one of claims 86 to 116, characterized in that the cationic film-forming polymer is present in a content ranging from 0.01% to 20% by weight, preferably from 0.01% to 15% by weight and even more preferentially from 0.05% to 5% by weight, relative to the total weight of the composition.

118. Process according to any one of claims 86 to 117, characterized in that the anionic film-forming polymer is present in a content ranging from 0.01% to 20% by weight, preferably from 0.05% to 15% by weight and even more preferentially from 0.1% to 7% by weight, relative to the total weight of the composition.

119. Process according to any one of claims 86 to 118, characterized in that the fatty phase comprises at least one wax.

120. Process according to claim 119, characterized in that the wax is chosen from the group formed by beeswax, lanolin wax, Chinese insect waxes, rice wax, carnauba wax, candelilla wax, ouricury wax, cork fibre wax, sugar cane wax, Japan wax, sumach wax, montan wax, microcrystalline waxes, paraffin waxes, ozokerites, ceresin wax, lignite wax, polyethylene waxes and the waxes obtained by Fisher-Tropsch synthesis, fatty acid esters of glycerides that are solid at 40° C., the waxes obtained by catalytic hydrogenation of animal or plant oils containing linear or branched C₈-C₃₂ fatty chains, silicone waxes and fluoro waxes, and mixtures thereof.

121. Process according to claim 119 or 120, characterized in that the wax is present in a content ranging from 0.1% to 50% by weight, preferably from 0.5% to 40% by weight and better still from 1% to 30% by weight, relative to the total weight of the composition.

122. Process according to any one of claims 86 to 121, characterized in that the fatty phase comprises at least one oil chosen from the group formed by hydrocarbon-based oils, fluoro oils and/or silicone oils of mineral, animal, plant or synthetic origin, alone or as a mixture.

123. Process according to any one of claims 86 to 122, characterized in that the fatty phase comprises at least one volatile oil.

124. Process according to any one of claims 86 to 123, characterized in that the fatty phase comprises a volatile oil chosen from hydrocarbon-based volatile oils containing from 8 to 16 carbon atoms.

125. Process according to claim 123 or 124, characterized in that the volatile oil is present in a content ranging from 0.1% to 98% by weight and preferably from 1% to 65% by weight, relative to the total weight of the composition.

126. Process according to any one of claims 86 to 125, characterized in that the composition comprises an aqueous phase containing water or a mixture of water and of water-miscible organic solvent.

127. Process according to any one of claims 86 to 126, characterized in that the composition contains at least one additive chosen from dyestuffs, surfactants, thickeners, antioxidants, fillers, preserving agents, fragrances, neutralizers and cosmetic or dermatological active agents, and mixtures thereof.

128. Process according to any one of claims 86 to 127, characterized in that the composition is in the form of a mascara, a product for the eyebrows or a product for the hair.
